(19) United States
(12) Patent Application Publication
(10) Pub. No.: US 2023/0172935 A1
(43) Pub. Date: Jun. 8, 2023

(54) METHODS OF MONITORING KRAS MUTATIONS

(71) Applicant: Cardiff Oncology, Inc., San Diego, CA (US)

(72) Inventors: Errin SAMUELSZ, San Diego, CA (US); Maya RIDINGER, San Diego, CA (US); Mark ERLANDER, San Diego, CA (US)

(21) Appl. No.: 17/923,803

(22) PCT Filed: May 6, 2021
(86) PCT No.: PCT/US2021/031192

§ 371 Date: Nov. 7, 2022

Related U.S. Application Data
(60) Provisional application No. 63/022,129, filed on May 8, 2020.

(51) Int. Cl. A61K 31/519; C12Q 1/6886; A61P 35/00; A61K 31/58; A61K 31/573; A61P 35/04; A61P 15/00

(52) U.S. Cl. CPC A61K 31/519; C12Q 1/6886; A61P 35/00; A61K 31/58; A61K 31/573; A61P 35/04; A61P 15/00; C12Q 2600/156; C12Q 2600/106; C12Q 1/6858

(57) ABSTRACT

Provided includes methods, compositions and kits for improving outcome of a cancer treatment, and methods, compositions and kits for determining responsiveness of a subject to a cancer treatment. The cancer treatment can comprise administering PLK1 inhibitor (e.g., onvansertib) to the subject.

### CROSS-REFERENCE TO RELATED APPLICATIONS

This application is a U.S. national phase application under 35 U.S.C. § 371 of International Application No. PCT/US2021/031192, filed on May 6, 2021 and published as WO 2021/226403 A1 on Nov. 11, 2021, which claims the benefit of priority to U.S. Patent Application No. 63/022,129, filed on May 8, 2020; the content of each of which is incorporated herein by reference in its entirety.

### BACKGROUND

### Field

The present application generally relates to cancer treatment. More specifically, methods for predicting and monitoring effectiveness of a cancer treatment are provided.

### Description of the Related Art

The Polo-like kinase 1 (PLK1) is the most well characterized member of the 5 members of the family of serine/threonine protein kinases and strongly promotes the progression of cells through mitosis. PLK1 performs several important functions throughout mitotic (M) phase of the cell cycle, including the regulation of centrosome maturation and spindle assembly, the removal of cohesins from chromosome arms, the inactivation of anaphase-promoting complex/cyclosome (APC/C) inhibitors, and the regulation of mitotic exit and cytokinesis. PLK1 plays a key role in centrosome functions and the assembly of bipolar spindles. PLK1 also acts as a negative regulator of p53 family members leading to ubiquitination and subsequent degradation of p53/TP53, inhibition of the p73/TP73 mediated pro-apoptotic functions and phosphorylation/degradation of bora, a cofactor of Aurora kinase A. During the various stages of mitosis PLK1 localizes to the centrosomes, kinetochores and central spindle. PLK1 is a master regulator of mitosis and aberrantly overexpressed in a variety of human cancers including AML and is correlated with cellular proliferation and poor prognosis. There is a need for methods for predicting/determining clinical benefits and outcome for cancer treatments involving PLK1 inhibitors.

### SUMMARY

Provided include methods, compositions and kits for determining responsiveness of a subject to a cancer treatment, methods, compositions and kits for improving outcome of a cancer treatment, and methods, compositions and kits for treating cancer.

Disclosed herein include methods of determining responsiveness of a subject to a cancer treatment. In some embodiments, a method of determining responsiveness of a subject to a cancer treatment, comprises treating a subject with cancer, the treating comprises administering a PLK1 inhibitor to the subject. The method comprises detecting change(s) in KRAS gene mutation(s) in the subject. The method comprises determining the responsiveness of the subject to the cancer treatment based on the change(s) detected in KRAS gene mutation(s).

In some embodiments, detecting change(s) in KRAS gene mutation(s) in the subject comprises detecting one or more mutations in KRAS gene in the subject (1) during the subject is treated for cancer, (2) before the subject is treated for cancer, (3) after the subject is treated for cancer, or a combination thereof. In some embodiments, detecting change(s) in KRAS gene mutation(s) in the subject comprises detecting KRAS gene mutations two or more times in the subject, and optionally at least two of the two or more times occur within 5, 7, 14, 28, or 35 days.

In some embodiments, change(s) in KRAS gene mutation(s) comprises (1) change(s) in KRAS gene mutation(s) during the subject is treated for cancer, (2) change(s) in KRAS gene mutation(s) from before the subject is treated for cancer to during the subject is treated for cancer, or a combination thereof. In some embodiments, detecting change(s) in KRAS gene mutation(s) comprises detecting variant allele frequency of KRAS gene. In some embodiments, the variant allele frequency is mutant allelic frequency (MAF). In some embodiments, KRAS gene mutation(s) is measured as the number (e.g., total number) of KRAS mutation alleles in a sample from the subject, and the change(s) in KRAS gene mutation(s) can be determined accordingly.

In some embodiments, the variant allele frequency of KRAS gene is determined by total mutation count, mean variant allele frequency, number of KRAS mutation alleles per ml of plasma, or a combination thereof. In some embodiments, detecting change(s) in KRAS gene mutation(s) in the subject comprises detecting change(s) in KRAS gene mutation(s) in a biological sample from the subject, or derivative thereof. In some embodiments, the biological sample comprises a bodily fluid, whole blood, plasma, one or more tissues, one or more cells, or a combination thereof. In some embodiments, the bodily fluid comprises blood, plasma, urine, or a combination thereof. In some embodiments, the biological sample comprises circulating tumor DNA (ctDNA), cell-free DNA (cfDNA), circulating tumor cell (CTC), or a combination thereof. In some embodiments, the method comprises analyzing the ctDNA using polymerase chain reaction (PCR) or next generation sequencing (NGS), and the PCR is optionally droplet digital PCR (ddPCR).

In some embodiments, the subject has one or more mutations in KRAS gene before being treated with the PLK1 inhibitor. In some embodiments, the subject does not have mutations in KRAS gene before being treated with the PLK1 inhibitor. In some embodiments, the cancer is a cancer associated with one or more KRAS mutations, for example lung cancer (e.g., non-small cell lung cancer), colorectal cancer, prostate cancer, or a combination thereof, that is associated with KRAS mutation.

In some embodiments, determining the responsiveness of the subject comprises determining if the subject is a responder of the treatment, if the subject is or is going to be in complete recover (CR), or if the subject is or is going to be in partial remission (PR). In some embodiments, determining the responsiveness of the subject comprises determining progression-free survival (PFS) of the subject. In some embodiments, determining the responsiveness of the subject comprises determining if the subject has a partial response to the treatment, if the subject has a complete response to the treatment, if the subject has a stable disease (SD) status, or if the subject has a progressive disease (PD) status.

In some embodiments, the KRAS mutation is measured by determining the amount of the KRAS mutations in the sample, determining the amount of the KRAS mutation in proportion to the amount of total KRAS in the sample, or both.

In some embodiments, the cancer treatment with the PLK1 inhibitor is maintained if the change in MAF of KRAS is a decrease of at least 25%, at least 50%, or at least 75%, and optionally the decrease is detected at the end of cycle 1 of the cancer treatment or at day 1 of cycle 2 of the cancer treatment. In some embodiments, the cancer treatment is for at least one month, at least three months, or at least six months. In some embodiments, the cancer treatment comprises a chemotherapy and the cancer treatment is modified to remove the chemotherapy partially or completely if the change in MAF of KRAS is a decrease of at least 50% or at least 75% after receiving the cancer treatment for six months. For example, the cancer treatment with onvansertib in combination of an additional cancer therapy (e.g., chemotherapy) can be maintained if the change in MAF of KRAS is a decrease of less than 75%; and the cancer treatment with onvansertib in combination of an additional cancer therapy (e.g., chemotherapy) can be modified by removing partially or completely the additional cancer therapy (e.g., chemotherapy).

In some embodiments, the method further comprises measuring KRAS mutation after partial or complete removal of the chemotherapy, and restoring the chemotherapy if the KRAS mutation level increases compared to the KRAS mutation level at the time of the removal of the chemotherapy. Measuring KRAS mutation can be, for example, 15 days, one month, two months, three months, six months, a year, two years, three years, or a range between any two of these values, after partial or complete removal of the chemotherapy. In some embodiments, the decrease is detected at the end of cycle 1 of the cancer treatment or at day 1 of cycle 2 of the cancer treatment.

In some embodiments, the cancer treatment with the PLK1 inhibitor is maintained if KRAS mutation in the samples decreases to below 0.01% or below 0.001% of KRAS in the sample. In some embodiments, the cancer treatment with the PLK1 inhibitor is modified or discontinued if the change in MAF of KRAS is a decrease of less than 50%, less than 25%, or less than 10%, and optionally the decrease is detected at the end of cycle 1 of the cancer treatment or at day 1 of cycle 2 of the cancer treatment. In some embodiments, the cancer treatment does not comprise a chemotherapy and the cancer treatment is modified to add a chemotherapy if the change in MAF of KRAS is a decrease of less than 50% or less than 75%. In some embodiments, the chemotherapy comprises irinotecan, and optionally the chemotherapy is FOLFIRI. In some embodiments, the cancer treatment with the PLK1 inhibitor is modified or discontinued if KRAS mutation in the samples does not decrease to below 0.01% or below 0.001% of KRAS in the sample.

In some embodiments, detecting change(s) in KRAS gene mutation(s) in the subject comprising detecting one or more KRAS mutations emerged in the subject after the subject being treated with the PLK1 inhibitor.

Disclosed herein include methods of improving outcome of a cancer treatment. In some embodiments, a method of improving outcome of a cancer treatment comprises detecting variant allele frequency of KRAS gene in a subject at a first time point in a first sample, the first time point is before the subject starts the cancer treatment, or during the cancer treatment, and the cancer treatment comprises administering a PLK1 inhibitor to the subject. The method comprises detecting variant allele frequency in KRAS gene in the subject at one or more additional time points in one or more additional samples after the subject, the at least one of the one or more additional time points is during the cancer treatment. The method comprises determining the difference of the variant allele frequency of KRAS between the first and the one or more additional samples, a decrease in the variant allele frequency in at least one of the one or more additional samples relative to the first sample indicates the subject as responsive to the cancer treatment. The method comprises continuing the cancer treatment to the subject if the subject is indicated as responsive to the cancer treatment, or discontinuing the cancer treatment to the subject and/or starting a different cancer treatment to the subject if the subject is not indicated as responsive to the cancer treatment. In some embodiments, the first time point is before the subject starts the cancer treatment. In some embodiments, at least two of the additional time points are during the cancer treatment.

Disclosed herein include methods of treating cancer. In some embodiments, a method of treating cancer comprises treating a subject with cancer, the treating comprises administering a PLK1 inhibitor to the subject. The method comprises determining a decrease, relative to a variant allele frequency of KRAS gene or number of KRAS mutant copy per unit in a first sample of the subject obtained at a first time point before the subject receives the cancer treatment or during the cancer treatment, in a variant allele frequency of KRAS gene in a second sample of the subject obtained at a second time point after the subject starts receiving the cancer treatment. The method comprises continuing with the cancer treatment.

In some embodiments, the first time point is prior or immediately prior to the cancer treatment. In some embodiments, the first time point is during the cancer treatment, and optionally at day 5, 7, 14, or 28 of the cancer treatment. In some embodiments, the one or more additional time points are during the cancer treatment, and optionally at day 5, 7, 14, 28, or 35 of the cancer treatment. In some embodiments, the first time point and at least one of the one or more additional time points are during the first cycle of the cancer treatment. In some embodiments, at least one of the one or more additional time points are during the first cycle of the cancer treatment, and at least one of the one or more additional time points are during the second cycle of the cancer treatment.

In some embodiments, the variant allele frequency is mutant allelic frequency (MAF). In some embodiments, the determining step comprises determining a decrease in the number of mutant copies per unit of the first sample and/or the second sample, the unit is optionally ml, and optionally the first sample and/or the second sample is a plasma sample. In some embodiments, the variant allele frequency of KRAS gene is determined by total mutation count, mean variant allele frequency, number of KRAS mutation alleles, or a combination thereof. In some embodiments, detecting variant allele frequency in KRAS gene comprises detecting variant allele frequency in KRAS gene in a biological sample from the subject, or derivative thereof.

In some embodiments, the biological sample comprises a bodily fluid, whole blood, plasma, one or more tissues, one or more cells, or a combination thereof. In some embodiments, the bodily fluid comprises blood, plasma, urine, or a combination thereof. In some embodiments, the biological sample comprises circulating tumor DNA (ctDNA), circulating tumor cell (CTC), or a combination thereof.

In some embodiments, the method comprises analyzing the ctDNA using polymerase chain reaction (PCR) or next generation sequencing (NGS), and the PCR is optionally droplet digital PCR (ddPCR).

In some embodiments, the subject has one or more mutations in KRAS gene before being treated with the PLK1 inhibitor. In some embodiments, the subject does not have mutations in KRAS gene before being treated with the PLK1 inhibitor. In some embodiments, the subject has received one or more prior cancer treatment.

In some embodiments, the cancer is advanced, metastatic, refractory, or relapsed. In some embodiments, the cancer is colorectal cancer, pancreatic cancer, leukemia, lung cancer, or a combination thereof. In some embodiments, the cancer is a KRAS mutation cancer. In some embodiments, the cancer is colorectal cancer, optionally metastatic colorectal cancer.

In some embodiments, the KRAS gene mutation(s) comprise mutations at codon 12, codon 13, codon 18, codon 61, codon 117, codon 146, or a combination thereof. In some embodiments, the KRAS gene mutation(s) comprise mutations at codon 12 and/or codon 13. In some embodiments, the KRAS gene mutation(s) comprise G12A, G12C, G12D, G12R, G12S, G12V, G13C, G13D, G13S, G13R, A18D, G61H, Q61L, Q61K, Q61R, K117N, A146T, A146V, A146P, A11V, or a combination thereof.

In some embodiments, the PLK1 inhibitor is onvansertib, BI2536, volasertib (BI 6727), GSK461364, HMN-176, HMN-214, AZD1775, CYC140, rigosertib (ON-01910), MLN0905, TKM-080301, TAK-960, Ro3280, or a combination thereof. In some embodiments, the PLK1 inhibitor is onvansertib.

The PLK1 inhibitor (e.g., onvansertib) can be administered to the subject under various schedule. For example, the subject can be administered with onvansertib in a continuous dosing schedule, or in a dosing schedule that the subject is given one or more days of break within a treatment cycle from the administration of the PLK inhibitor. In some embodiments, the treatment comprises administration of onvansertib every day in a cycle of about 28 days. In some embodiments, the treatment comprises administration of onvansertib for the first 21 days and not the last 7 days in a cycle of 28 days. In some embodiments, the treatment comprises administration of onvansertib for the first 14 days and not the last 14 days in a cycle of 28 days. In some embodiments, the treatment comprises administration of onvansertib for ten days or fourteen days in a cycle of 28 days. In some embodiments, the treatment comprises administration of onvansertib for five days in the first 14 days and five days in the second 14 days in a cycle of 28 days. In some embodiments, the treatment comprises administration of onvansertib for seven days (e.g., day 1 to day 7) in the first 14 days and seven days (e.g., day 15 to day 21) in the second 14 days in a cycle of 28 days. In some embodiments, the treatment comprises administration of onvansertib at 6 mg/m²-24 mg/m², optionally 6 mg/m²-12 mg/m² or 12 mg/m²-18 mg/m². In some embodiments, a maximum concentration (Cₘₐₓ) of onvansertib in a blood of the subject is from about 100 nmol/L to about 1500 nmol/L.

In some embodiments, an area under curve (AUC) of a plot of a concentration of onvansertib in a blood of the subject over time is from about 1000 nmol/L·hour to about 400000 nmol/L·hour. In some embodiments, a time (Tmax) to reach a maximum concentration of onvansertib in a blood of the subject is from about 1 hour to about 5 hours. In some embodiments, an elimination half-life (T_{1/2}) of onvansertib in a blood of the subject is from about 10 hours to about 60 hours.

In some embodiments, the cancer treatment comprises administering to the subject at least one additional cancer therapeutics or cancer therapy. In some embodiments, the additional cancer therapeutics comprises FOLFIRI, bevacizumab, abiraterone, FOLFOX, an anti-EGFR agent, a KRAS directed inhibitor, gemcitabine, abraxane, nanoliposomal irinotecan, 5-FU, or a combination thereof; the anti-EGFR agents is optionally cetuximab, and KRAS directed inhibitor is optionally a G12C inhibitor, a G12D inhibitor or a combination thereof. In some embodiments, the PLK inhibitor and the cancer therapeutics or cancer therapy are co-administered simultaneously or sequentially.

In some embodiments, the cancer treatment comprises one or more cycles, and change(s) in KRAS gene mutation(s) or variant allele frequency of KRAS is detected before, during and/or after each cycle of the cancer treatment. In some embodiments, each cycle of treatment is at least 21 days. In some embodiments, each cycle of treatment is from about 21 days to about 28 days. In some embodiments, the subject is human.

Use of a PLK1 inhibitor as a treatment of a subject with cancer, the responsiveness of the subject to the treatment is determined using a method the treatment outcome is improved using a method the subject is treated using a method the use the PLK1 inhibitor is onvansertib.

Disclosed herein include embodiments of use of a PLK1 inhibitor as a treatment of a subject with cancer. In some embodiments, the responsiveness of the subject to the treatment is determined using any method of the present disclosure. In some embodiments, the treatment outcome is improved using any method of the present disclosure. In some embodiments, the subject is treated using any method of the present disclosure. In some embodiments, the PLK1 inhibitor is onvansertib.

As disclosed herein, periodic measurement of a KRAS mutation in cell-free DNA in a bodily fluid of a cancer patient can guide treatment decisions. Provided herein include a method comprising: (a) treating a patient for a cancer characterized by a KRAS mutation and (b) periodically sampling a bodily fluid from the patient and measuring the KRAS mutation in cell-free DNA (cfDNA) in the bodily fluid.

Also provided includes a method comprising (a) treating a patient for colorectal cancer characterized by a KRAS mutation, and (b) periodically sampling a bodily fluid from the patient and measuring the KRAS mutation in cell-free DNA in the bodily fluid, wherein the treatment comprises administration of a PLK1 inhibitor.

### BRIEF DESCRIPTION OF THE DRAWINGS

FIG. 1 is an illustration of the synthetic lethality of a PLK1 inhibitor against a cancer cell having a KRAS mutant.

FIG. 2 is an illustration of signal transduction pathways directing mitosis.

FIG. 3 is graphs showing mutant KRAS in cell-free plasma DNA of six patients being treated with onvansertib with FOLFIRI and bevacizumab. Arrows show the first time point with undetectable mutant KRAS.

FIG. 4A is a graph showing radiographic response of five patients treated with onvansertib at 12 mg/m² with FOLFIRI and bevacizumab. FIG. 4B is a graph showing the durability of response of seven patients treated with onvansertib at 12 mg/m² with FOLFIRI and bevacizumab.

FIG. 5 is a graph showing cell viability in onvansertib-treated KRAS mutant and WT isogenic CRC cells.

FIG. 6A and FIG. 6B are graphs showing anti-tumor activity of onvansertib in combination with irinotecan and 5-FU in the HCT-116 KRAS-mutant CRC xenograft model.

FIG. 7 shows treatment schedule for onvansertib in combination of FOLFIRI and bevacizumab.

FIG. 8A is a graph showing treatment response and duration. FIG. 8B is a graph showing radiographic response.

FIG. 9 shows graphs for KRAS mutation MAF and changes in tumor size from baseline in two patients.

FIG. 10A is a graph showing treatment response and duration. FIG. 10B is a graph showing changes in tumor size from baseline.

FIG. 11A is a graph showing % KRAS MAF changes after 1 cycle. FIG. 11B is a graph showing KRAS MAF changes over time.

FIG. 12 shows PFS of EAP participants with detectable plasma KRAS mutant at baseline.

FIG. 13 shows a participant's treatment history.

FIG. 14A shows the baseline, 8-week and 16-week scans for the participant with treatment history shown in FIG. 13. FIG. 14B shows decreases in tumor lesions accompanied by a decrease in KRAS MAF in the participant with treatment history shown in FIG. 13.

FIG. 15 shows treatment histories of two participants to EAP.

FIG. 16 shows treatment schedule for the study described in Example 3.

FIG. 17 shows efficacy of the study described in Example 3.

FIG. 18 shows gene differentially mutated in SD and PD patients.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e., Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place. In some embodiments, the patient is a human or an animal. In some embodiments, the patient is a mammal.

As used herein, “administration” or “administering” refers to a method of giving a dosage of a pharmaceutically active ingredient to a vertebrate.

As used herein, a “dosage” refers to the combined amount of the active ingredients (e.g., cyclosporine analogues, including CRV431).

As used herein, a “unit dosage” refers to an amount of therapeutic agent administered to a patient in a single dose.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “prophylaxis,” “prevent,” “preventing,” “prevention,” refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, each of the terms “partial response” and “partial remission” refers to the amelioration of a cancerous state, as measured by, for example, tumor size and/or cancer marker levels, in response to a treatment. In some embodiments, a “partial response” means that a tumor or tumor-indicating blood marker has decreased in size or level by about 50% in response to a treatment. The treatment can be any treatment directed against cancer, including but not limited to, chemotherapy, radiation therapy, hormone therapy, surgery, cell or bone Marrow transplantation, and immunotherapy. The size of a tumor can be detected by clinical or by radiological means. Tumor-indicating markers can be detected by means well known to those of skill, e.g., ELISA or other antibody-based tests.

As used herein, each of the terms “complete response” or “complete remission” means that a cancerous state, as measured by, for example, tumor size and/or cancer marker levels, has disappeared following a treatment, including but are not limited to, chemotherapy, radiation therapy, hormone therapy, surgery, cell or bone marrow transplantation, and immunotherapy. The presence of a tumor can be detected by clinical or by radiological means. Tumor-indicating markers can be detected by means well known to those of skill, e.g., ELISA or other antibody-based tests. A “complete response” does not necessarily indicate that the cancer has been cured, however, as a complete response can be followed by a relapse.

The abbreviations shown below are used herein:
PR=partial response
SD=stable disease
PD=progressive disease;
CXD1=Cycle X Day 1
PFS=progression-free survival

Disclosed herein include methods, compositions and kits for determining responsiveness of a subject to a cancer treatment, methods, compositions and kits for improving outcome of a cancer treatment, and methods, compositions and kits for treating cancer.

### KRAS

The KRAS gene (also known as Kirsten rat sarcoma viral oncogene homolog, KRAS Proto-Oncogene, GTPase, K-Ras, KRAS2) is a proto-oncogene that encodes a GTPase that is part of signal transduction pathways that regulate mitosis.

Several mutations in KRAS activate the protein and are implicated in cancer such as acute myelogenous leukemia (AML), juvenile myelomonocytic leukemia (JMML), gastric cancer, colorectal cancer, pancreatic cancer and lung cancer. Cancers with mutant KRAS often have aggressive growth. Mutations in the KRAS genes have been found codon 12, codon 13, codon 18, codon 61, codon 117, and codon 146. The most common activating mutations in the KRAS gene are found in codons 12 and 13, including but not limited to G13D, G13D, G12V, G12D, G12A, G12R, G12S and G12C. Non-limiting examples of KRAS mutations include A18D, Q61H, and K117N. As used herein, KRAS gene mutations can comprise, for example, G12A, G12C, G12D, G12R, G12S, G12V, G13C, G13D, G13S, G13R, A18D, G61H, Q61L, Q61K, Q61R, K117N, A146T, A146V, A146P, A11V, or a combination thereof.

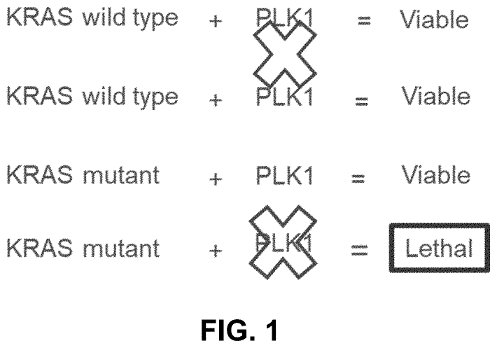

Drugs are in development that target KRAS G12C, but no drugs are currently available that target KRAS activated by the other mutations. Efforts to target cancers with KRAS mutations focus on inhibiting proteins that share the same signal transduction pathways as KRAS. A genome-wide RNAi screen was completed to identify what gene(s) is necessary for KRAS-mutated tumor cells to drive tumor growth. Among the genes identified was PLK1, where inhibition of PLK1 was hypothesized to be a synthetic lethal against cancers with KRAS mutants. Synthetic lethality is when a combination of deficiencies in the expression of two or more genes leads to cell death, whereas a deficiency in only one of these genes does not. In the current context, as illustrated in FIG. 1, when PLK1 is inhibited in a cell with a wild-type KRAS, e.g., a non-cancerous cell, the cell remains viable. However, cell death occurs when PLK1 is inhibited in a cancer cell with a KRAS mutant.

Tumors having KRAS mutations and resistant to treatment can emerge during treatment. This resistance is particularly common with anti-EGFR treatment of metastatic colorectal cancer (mCRC), where up to 50% of mCRC patients undergoing standard-of-care anti-EGFR treatment (e.g., cetuximab and/or panitumumab) with wild-type KRAS develop resistant tumors with KRAS mutations. Secondary treatment options are needed for those patients. There is also a need for a KRAS assay that can enable rapid assessment of the presence of prevalence of mutant KRAS as an early predictor of response to treatment. Methods, compositions and kits disclosed herein can be used for treating cancer, for example colorectal cancer. Colorectal cancer (CRC) is often associated with a KRAS mutation. The standard-of-care chemotherapy for colorectal cancer is currently FOLFIRI, which is a combination of leucovorin (folinic acid), 5-fluorouracil (5-FU), and irinotecan. Bevacizumab is often combined with FOLFIRI, however, that combination has only a 4% response rate against metastatic CRC (mCRC).

### PLK Inhibitors, Dosing and Pharmacokinetics

Polo-like kinases (PLK) are a family of five highly conserved serine/threonine protein kinases. PLK1 is a master regulator of mitosis and is involved in several steps of the cell cycle, including mitosis entry, centrosome maturation, bipolar spindle formation, chromosome separation, and cytokinesis. PLK1 has been shown to be overexpressed in solid tumors and hematologic malignancies. PLK1 inhibition induces G2-M-phase arrest with subsequent apoptosis in cancer cells, and has emerged as a promising targeted therapy. Non-limiting examples of PLK1 inhibitor include onvansertib, B12536, volasertib (BI 6727), GSK461364, HMN-176, HMN-214, AZD1775, CYC140, rigosertib (ON-01910), MLN0905, TKM-080301, TAK-960, Ro3280, and any combination thereof.

Onvansertib (also known as PCM-075, NMS-1286937, NMS-937, “compound of formula (I)” described in U.S. Pat. No. 8,927,530; IUPAC name 1-(2-hydroxyethyl)-8-{[5-(4-methylpiperazin-1-yl)-2-(trifluoromethoxy) phenyl]amino}-4,5-dihydro-1H-pyrazolo[4,3-h] quinazoline-3-carboxamide) is a selective ATP-competitive PLK1 inhibitor. Biochemical assays demonstrated high specificity of onvansertib for PLK1 among a panel of 296 kinases, including other PLK members. Onvansertib has potent in vitro and in vivo antitumor activity in models of both solid and hematologic malignancies. For example, it shows high potency in proliferation assays having low nanomolar activity on a large number of cell lines, both from solid as well as hematologic tumors. Onvansertib is the first PLK1 specific ATP competitive inhibitor administered by oral route to enter clinical trials with proven antitumor activity in different preclinical models.

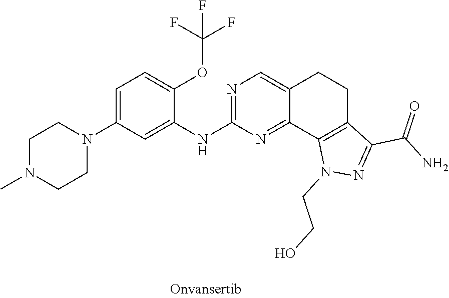 [SMILES: CN1CCN(c2ccc(OC(F)(F)F)c(Nc3ncc4c(n3)-c3c(c(C(N)=O)nn3CCO)CC4)c2)CC1]

Onvansertib potently causes a mitotic cell-cycle arrest followed by apoptosis in cancer cell lines and inhibits xenograft tumor growth with a clear PLK1-related mechanism of action at well tolerated doses in mice after oral administration. In addition, onvansertib shows activity in combination therapy with approved cytotoxic drugs, such as irinotecan, in which there is enhanced tumor regression in HT29 human colon adenocarcinoma xenografts compared to each agent alone, and shows prolonged survival of animals in a disseminated model of AML in combination therapy with cytarabine. Onvansertib has favorable pharmacologic parameters and good oral bioavailability in rodent and nonrodent species, as well as proven antitumor activity in different nonclinical models using a variety of dosing regimens, which may potentially provide a high degree of flexibility in dosing schedules, warranting investigation in clinical settings. Onvansertib has several advantages over previous PLK inhibitors, including high selectivity for PLK1 only, oral availability and half-life of about 24 hours.

A Phase 1 dose-escalation study with onvansertib has been conducted in adult subjects with advanced/metastatic solid tumors at a single study site in the U.S. The primary objective of that study was to determine a maximum tolerated dose (MTD) of onvansertib in adult subjects with advanced/metastatic solid tumors. Secondary objectives of the study were to define antitumor activity. In that study, a recommended phase 2 dose of 24 mg/m² was established and 5 of 16 evaluable patients had stable disease.

A phase I, first-in-human, dose-escalation study of onvansertib in patients with advanced/metastatic solid tumors identified neutropenia and thrombocytopenia as the primary dose-limiting toxicities. These hematologic toxicities were anticipated on the basis of the mechanism of action of the drug and were reversible, with recovery occurring within 3 weeks. The half-life of onvansertib was established between 20 and 30 hours. The oral bioavailability of onvansertib plus its short half-life provide the opportunity for convenient, controlled, and flexible dosing schedules with the potential to minimize toxicities and improve the therapeutic window. Pharmacodynamics and biomarker studies, including baseline genomic profiling, serial monitoring of mutant allele fractions in plasma, and the extent of PLK1 inhibition in circulating blasts, have been performed to identify biomarkers associated with clinical response and are described in PCT Application No. PCT/US2021/013287 titled “Circulating Tumor DNA as a Biomarker for Leukemia Treatment” and filed on Jan. 13, 2021, the content of which is incorporated herein by reference in its entirety.

The cancer treatment of the present disclosure can comprise administration of a PLK1 inhibitor (e.g., onvansertib) to a subject with cancer for a desired duration in a cycle, two cycles, or more cycles. The desired duration in each cycle can independently be one, two, three, four, five, six, seven, eight, nine, ten, or more days. The cycle can be, for example, at least 20 days, 21 days, 22 days, 23 days, 24 days, 25 days, or more, in length. For example, a single cycle of the treatment can comprise administration of the PLK1 inhibitor (e.g., onvansertib) for four days, five days, six days, seven days, eight days, nine days, ten days, eleven days, twelve days, thirteen days, fourteen days, fifteen days, sixteen days, seventeen days, eighteen days, nineteen days, twenty days, or more in a cycle (e.g., a cycle of at least 21 days (e.g., 21 to 28 days)). In some embodiments, the treatment can comprise administration of the PLK1 inhibitor (e.g., onvansertib) for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the PLK1 inhibitor (e.g., onvansertib) in a single cycle of the treatment can be continuous or with one or more intervals (e.g., one day or two days of break). In some embodiments, the treatment comprises administration of the PLK1 inhibitor (e.g., onvansertib) for five days in a cycle of 21 to 28 days. In some embodiments, the duration of administration of the PLK1 inhibitor in one cycle can be different from the duration of the administration of the PLK1 inhibitor in one or more other cycles. For example, the PLK1 inhibitor can be administered to the subject for 10 days (e.g., day 1 to day 5 in the first 14 days and day 1 to day 5 in the last 14 days in a 28-day cycle) for the first cycle, and for 14 days in the second cycle (e.g., day 1 to day 7 in the first 14 days and day 1 to day 7 in the last 14 days in a 28-day cycle). The length of each of the cycles can vary. For example, cycle 1 can be 28 days, and cycle 2 can be 21 days.

The cancer treatment disclosed herein can comprise administration of the PLK1 inhibitor (e.g., onvansertib) at, or at about, 12 mg/m²-90 mg/m², for example, as a daily dose. For example, the treatment can comprise daily administration of the PLK1 inhibitor (e.g., onvansertib) at, or at about, 8 mg/m², 10 mg/m², 12 mg/m², 14 mg/m², 15 mg/m², 16 mg/m², 18 mg/m², 20 mg/m², 23 mg/m², 27 mg/m², 30 mg/m², 35 mg/m², 40 mg/m², 45 mg/m², 50 mg/m², 55 mg/m², 60 mg/m², 65 mg/m², 70 mg/m², 80 mg/m², 85 mg/m², 90 mg/m², a range between any two of these values, or any value between 8 mg/m²-90 mg/m². In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the daily dose of the PLK1 inhibitor (e.g., onvansertib) is 12 mg/m², 15 mg/m², 18 mg/m², or 24 mg/m². In some embodiments, the daily dose of the PLK1 inhibitor (e.g., onvansertib) is 15 mg/m². The daily dose of the PLK1 inhibitor (e.g., onvansertib) for each cycle of treatment can vary. For example, the daily dose of the PLK1 inhibitor (e.g., onvansertib) for the first cycle can be 12 mg/m², and the daily dose of the PLK1 inhibitor (e.g., onvansertib) for the second cycle can be increased to, for example, 15 mg/m². In some embodiments, the daily dose of the PLK1 inhibitor (e.g., onvansertib) for the second cycle can then be increased to, for example, 18 mg/m².

A maximum concentration (Cₘₐₓ) of the PLK1 inhibitor (e.g., onvansertib) in a blood of the subject (during the treatment or after the treatment) when the PLK1 inhibitor is administered alone or in combination with one or more additional cancer therapeutics (e.g., FOLFIRI and bevacizumab) can be from about 100 nmol/L to about 1500 nmol/L. For example, the C. of the PLK1 inhibitor (e.g., onvansertib) in a blood of the subject when the PLK1 inhibitor is administered alone or in combination with one or more additional cancer therapeutics (e.g., FOLFIRI and bevacizumab) can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with one or more additional cancer therapeutics (e.g., FOLFIRI and bevacizumab) can be from about 1000 nmol/L·hour to about 400000 nmol/L·hour. For example, the AUC of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with one or more additional cancer therapeutics (e.g., FOLFIRI and bevacizumab) can be, or be about, 1000 nmol/L·hour, 5000 nmol/L·hour, 10000 nmol/L·hour, 15000 nmol/L·hour, 20000 nmol/L·hour, 25000 nmol/L·hour, 30000 nmol/L·hour, 35000 nmol/L·hour, 40000 nmol/L·hour, a range between any two of these values, or any value between 1000 nmol/L·hour and 400000 nmol/L·hour.

A time (Tₘₐₓ) to reach a maximum concentration of the PLK1 inhibitor (e.g., onvansertib) in a blood of the subject when the PLK1 inhibitor is administered alone or in combination with one or more additional cancer therapeutics (e.g., FOLFIRI and bevacizumab) can be from about 1 hour to about 5 hours. For example, the time (Tₘₐₓ) to reach a maximum concentration of the PLK1 inhibitor (e.g., onvansertib) in a blood of the subject when the PLK1 inhibitor is administered alone or in combination with the one or more additional cancer therapeutics (e.g., FOLFIRI and bevacizumab) can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of the PLK1 inhibitor (e.g., onvansertib) in a blood of the subject when the PLK1 inhibitor is administered alone or in combination with one or more additional cancer therapeutics (e.g., FOLFIRI and bevacizumab) can be from about 10 hours to about 60 hours. For example, the elimination half-life (T_{1/2}) of the PLK1 inhibitor (e.g., onvansertib) in a blood of the subject when the PLK1 inhibitor is administered alone or in combination with one or more additional cancer therapeutics (e.g., FOLFIRI and bevacizumab) can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

### Detection of KRAS Mutations

KRAS mutations can be detected in a biological sample (including but not limited to a bodily fluid (e.g., a blood sample)) from a subject of interest (e.g., a subject with colorectal cancer, a subject is in partial remission of colorectal cancer, or a subject suspected to have colorectal cancer). For example, the mutations can be detected in the circulating tumor cells (CTCs), circulating tumor DNA (ctDNA), PBMC, or a combination thereof, obtained from plasma fraction, serum fraction, or both, of a blood sample. In some embodiments, the bodily sample is whole blood, serum, plasma, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine, or any combination thereof. In some embodiment, the CTCs and/or ctDNA is obtained from blood and fractions thereof. A sample can be in the form originally isolated from a subject or can have been subjected to further processing to remove or add components, such as cells, or enrich for one component relative to another. Thus, it can be advantageous in some embodiments to analyze plasma or serum containing ctDNA. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may not have cancer or a detectable cancer symptom. The subject may have been treated with one or more cancer therapy, e.g., any one or more of chemotherapies, antibodies, vaccines or biologics. The subject may be in remission. The subject may be suspected to have cancer or any cancer-associated genetic mutations/disorders.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., ctDNA. Others are released from healthy cells. cfDNA can be obtained from a bodily fluid without the need to perform an in vitro cell lysis step, and thus presents a non-invasive option for genomic analysis. Provided herein include methods, compositions, kits and systems for detecting and/or analyzing cell free nucleic acids (e.g., ctDNA) in bodily fluid (e.g., peripheral blood) for clinical outcome prediction/determination. The methods can comprise combined analysis of single cells and cell-free nucleic acids. Provided herein include methods utilizing ctDNA from whole blood (e.g., plasma and/or serum) for therapeutic monitoring, and minimal/molecular residual disease determination.

Various assays (e.g., sequencing assays) can be used to detect and analyze ctDNA or nucleic acids from CTCs. The methods provided herein can comprise isolation and analysis of ctDNA from the blood (e.g., plasma and/or serum) of a subject of interest (e.g., a subject with colorectal cancer), employing the use of molecular barcoding and sequencing as a readout. The method can comprise isolating plasma and ctDNA from intact cell-depleted blood. The method can comprise centrifugation to generate plasma and extraction of nucleic acids from plasma, followed by library prep with barcoding, sequencing, and then analysis. The ctDNA, for example, can be obtained from a plasma sample by known methods, and can be analyzed by methods including but not limited to polymerase chain reaction (PCR) and next generation sequencing (NGS). In some embodiments, the ctDNA is analyzed using droplet digital PCR (ddPCR).

The ctDNA can carry one or more types of mutations, for example, germline mutations, somatic mutations, or both. Germline mutations refer to mutations existing in germline DNA of a subject. The ctDNA from a subject can carry one or more mutations in one or more genes, for example KRAS mutations. Somatic mutations refer to mutations originating in somatic cells of a subject, e.g., cancer cells. In some embodiments, the mutation can be a colorectal cancer-associated KRAS mutation.

Exemplary amounts of ctDNA in a biological sample (e.g., plasma or serum) before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of ctDNA molecules. The method can comprise obtaining 1 femtogram (fg) to 200 ng ctDNA. The ctDNA can have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

ctDNA can be isolated from bodily fluids (e.g., plasma) through a fractionation or partitioning step in which ctDNA, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. After such processing, samples can include various forms of nucleic acid including double stranded DNA and single stranded DNA. In some embodiments, single stranded DNA can be converted to double stranded forms so they are included in subsequent processing and analysis steps.

There are provided, in some embodiments, methods, reagents, compositions, and systems for analyzing complex genomic material while reducing or eliminating loss of molecular characteristic (e.g., epigenetic or other types of structural) information that is initially present in the complex genomic material. In some embodiments, molecular tags can be used to track ctDNA and determine genetic modifications (e.g., SNVs, indels, gene fusions and copy number variations). The method for detecting and analyzing ctDNA can comprise: classifying one or more variant properties derived from the sequence reads generated from one or more sequencing assays on the isolated cfNA as a true cancer-associated variant, a clonal hematopoiesis of indeterminate potential (CHIP)-associated variant, and/or a mutation of unknown origin. The method can comprise: adjusting the prediction score, the MRD score, and/or the efficacy score based on the classification of the one or more variant properties derived from the sequence reads generated from one or more sequencing assays on the isolated ctDNA.

Methods, compositions, kits and systems disclosed herein can be applied to different types of subjects. For example, the subject can be a subject receiving a cancer treatment, a subject at cancer remission e.g., partial remission), a subject has received one or more cancer treatment, or a subject suspected of having cancer. The subject can have a stage I cancer, a stage II cancer, a stage III cancer, and/or a stage IV cancer. The cancer can comprise a solid cancer, for example colorectal cancer, including metastatic colorectal cancer (mCRC). The cancer can be KRAS-mutated or not KRAS-mutated. The methods disclosed herein can comprise: administering a therapeutic intervention to the subject. The therapeutic intervention can comprise a different therapeutic intervention, an antibody, an adoptive T cell therapy, a chimeric antigen receptor (CAR) T cell therapy, an antibody-drug conjugate, a cytokine therapy, a cancer vaccine, a checkpoint inhibitor, radiation therapy, surgery, a chemotherapeutic agent, or any combination thereof. The therapeutic intervention can be administered at a time when the subject has an early-stage cancer, and wherein the therapeutic intervention is more effective that if the therapeutic intervention were to be administered to the subject at a later time.

As disclosed herein, useful information such as the effectiveness and/or clinical benefits of the cancer treatment can be obtained by evaluating ctDNA from more than one plasma sample, for example plasma collected (a) before or at treatment, and (b) at least once after treatment has started. In those embodiments, the second or subsequent samples can be taken at any time after treatment has started, e.g., after the first round of treatment, after multiple rounds of treatment, or after the colorectal cancer is no longer detected in order to determine whether the colorectal cancer has returned. See Example, where multiple plasma samples were evaluated in conjunction with bone marrow and peripheral blood cells. In some embodiments, the blood sample (e.g., plasma) collected after treatment is collected after a first round of treatment, e.g., at least 10, 15, 20, 21, 28, or 35 days, or any number of days in between or outside of those numbers, after the start of treatment.

Analyzing ctDNA can comprises analyzing ctDNA for one or more markers (e.g., ctDNAs comprising variant/mutant alleles). For example, ctDNA can be analyzed to assess variant allele frequency (VAF), change in the mean VAF, total mutation burden, and/or development of new KRAS mutations in a subject with cancer (e.g., colorectal cancer). The subject can a subject to be selected for a cancer treatment, a subject that is undergoing a cancer treatment, or a subject that has undergone a cancer treatment. In some embodiments, the ctDNA analysis measures the amount of a KRAS mutation in the ctDNA. In some embodiments, the ctDNA analysis measures mutation allelic frequencies (MAF) of KRAS gene in the ctDNA. For the methods disclosed herein, analyzing ctDNA from a subject can comprise detecting variant allele frequency (VAF) in the ctDNA, and a change in VAF at different time points can indicate the subject as responsive to the cancer treatment.

As described herein, a decrease in MAF during treatment, e.g., when comparing the MAF before treatment with the MAF after the first cycle of treatment, is indicative or predictive of clinical response. For example, a decrease in ctDNA MAF of KRAS can be indicative of a positive clinical outcome. The decrease in MAF of KRAS can be 25%, 30%, 35%, 40%, 45%, 50%, 55%, 60%, 65%, 70%, 75%, 80%, or a number or a range between any two of these values. The decrease in MAF of KRAS can be at least, or at least about, 25%, 30%, 35%, 40%, 45%, 50%, 55%, 60%, 65%, 70%, 75%, 80%. MAF determination can be used in the methods, compositions, kits and systems described herein for ctDNA analysis herein to determine effectiveness of treatment and future treatment of cancer (e.g., colorectal cancer). In some embodiments, these methods can be used to decide whether to continue a treatment, e.g., when there is a decrease in MAF during the treatment, or change the treatment, e.g., if there is not a decrease in MAF during treatment.

Methods for Determining Efficacy and/or Improving Outcome for Cancer Treatment

Disclosed herein include methods, compositions, kits, and systems for predicting/determining clinical outcome for a cancer treatment, monitoring of the cancer treatment, predicting/determining responsiveness of a subject to the cancer treatment, determining the cancer status of a subject, and improving cancer treatment outcome. The treatment comprises administering, a PLK1 inhibitor (e.g., onvansertib) to the subject. For example, the treatment can be a combination treatment using a PLK1 inhibitor (e.g., onvansertib) and one or more cancer therapeutics (e.g., FOLFIRI and bevacizumab).

The methods, compositions, kits and systems can be used to guide the cancer treatment, provide treatment recommendations, reduce or avoid unnecessary ineffective treatment for patients. For example, ctDNA and/or CTCs can be analyzed to predict/determine clinical outcome for cancer treatment comprising administration of a PLK1 inhibitor of the present disclosure, monitor the combination treatment, predict/determine responsiveness of a subject to the treatment, determine cancer status in a subject, improve cancer treatment outcome, guide cancer treatment, provide cancer treatment recommendations, and/or to reduce or avoid ineffective cancer treatment. ctDNA can be analyzed to predict/determine clinical outcome for cancer treatment, monitor cancer treatment, predict/determine responsiveness of a subject to a cancer treatment, determine cancer status in a subject, improve cancer treatment outcome, guide cancer treatment, provide treatment recommendations, and/or to reduce or avoid ineffective cancer treatment. Such analysis of ctDNA has been described in PCT Application No. PCT/US2021/013287, the content of which is incorporated herein by reference in its entirety.

The first time point for determining KRAS gene mutation (e.g., determining variant allele frequency of KRAS gene) can be, for example, prior or immediately prior, to the cancer treatment (that is, pre-dosing). The at least one of the one or more additional time points can be, for example, at the end, or right before the end, of or after at least a cycle (e.g. the first cycle, the second cycle, the third cycle, or any of the subsequent cycles) of the cancer treatment. In some embodiments, the cycle of the cancer treatment is the first cycle of the cancer treatment. In some embodiments, the first time point is prior or immediately prior to a first cycle of the cancer treatment. In some embodiments, the one or more additional time points are at the end, or right before the end, of a second cycle, a third cycle, a fourth cycle, and/or a fifth cycle of the treatment. In some embodiments, the one or more additional time points are after a second cycle, a third cycle, a fourth cycle, and/or a fifth cycle of the cancer treatment. In some embodiments, the first cycle of the cancer treatment is immediately prior (e.g., one day, two days, three days, four days, or five days) to the second cycle of the cancer treatment. In some embodiments, the method comprises continuing the cancer treatment to the subject if the subject is indicated as responsive to the cancer treatment. In some embodiments, the method comprises discontinuing the cancer treatment to the subject and/or starting a different cancer treatment to the subject if the subject is not indicated as responsive to the cancer treatment.

In some embodiments, the first time point is prior or immediately prior to the onset of the cancer treatment (e.g., a combination treatment), and at least one of the one or more additional time points are at the end of or after at least a cycle of the treatment. In some embodiments, the cycle of the combination treatment is the first cycle of the treatment. In some embodiments, the first time point is prior or immediately prior to a first cycle of the treatment, and the one or more additional time points are at the end of or after a second cycle of the treatment. In some embodiments, the first cycle of the combination treatment is immediately prior to the second cycle of the treatment. In some embodiments, the method comprises continuing the treatment to the subject if the subject is indicated as responsive to the treatment. In some embodiments, the method comprises discontinuing the combination treatment to the subject and/or starting a different treatment to the subject if the subject is not indicated as responsive to the treatment.

The first sample can comprise ctDNA and/or CTCs from the subject at various time points, for example before the treatment or during the treatment. In some embodiments, the first sample comprises ctDNA and/or CTCs from the subject before the treatment (for example, immediately before the first cycle of the treatment). In some embodiments, the first sample comprises ctDNA and/or CTCs from the subject before the second cycle of the treatment (for example, after the completion of the first cycle of the treatment and immediately before the second cycle of the treatment). The additional samples can comprise ctDNA and/or CTCs from the subject during and/or after the treatment. In some embodiments, the additional samples comprise ctDNA from the subject right before the end of and/or after the treatment. In some embodiments, the additional samples comprise ctDNA and/or CTCs from the subject right before the end of and/or after the first cycle, the second cycle, the third cycle, the fourth cycle, and/or the fifth cycle of the treatment.

Some embodiments comprises detecting variant allele frequency of KRAS mutation(s) in the ctDNA. In some embodiments, analyzing the ctDNA comprises detecting variant allele frequency in the ctDNA obtained from the subject at a first time point in a first sample, detecting variant allele frequency of KRAS gene in the ctDNA obtained from the subject at one or more additional time points in one or more additional samples, and determining the difference of the variant allele frequency in ctDNA between the first and at least one of the one or more additional samples, an increase in the variant allele frequency of KRAS at the additional sample(s) relative to the first sample indicates that the subject is not a responder for the cancer treatment. In some embodiments, the method comprises discontinuing the cancer treatment and/or starting an additional cancer treatment to the subject if the subject is indicated as a non-responder to the cancer treatment. The additional treatment can be the same or different from the current or prior treatment.

The variant allele frequency of KRAS mutation(s) in ctDNA can be determined, for example, by total mutation count of KRAS mutation(s) in the ctDNA in each of the first sample and one or more additional samples, or by the mean variant allele frequency of KRAS mutation(s) in each of the first sample and one or more additional samples, or by the number of KRAS mutation alleles per ml in each of the first sample and one or more additional samples (e.g., plasma samples). The ctDNA can be analyzed using, for example, PCR, next generation sequencing (NGS), and/or droplet digital PCR (ddPCR). The sample disclosed herein can be derived from, for example, whole blood of the subject, plasma of the subject, serum of the subject, or a combination thereof. In some embodiments, the ctDNA is from whole blood of the subject, plasma of the subject, serum of the subject, or a combination thereof.

In some embodiments, the method comprises analyzing ctDNA of the subject before the treatment. In some embodiments, the treatment comprises one or more cycles, and the ctDNA is analyzed before, during and after each cycle of the treatment. Each cycle of treatment can be at least 21 days. In some embodiments, each cycle of treatment is from about 21 days to about 28 days. In some embodiments, the subject is human.

Disclosed herein include a method of determining responsiveness of a subject to a cancer treatment, comprising treating a subject with cancer and the treating comprises administering a PLK1 inhibitor to the subject; detecting change(s) in KRAS gene mutation(s) in the subject, and determining the responsiveness of the subject to the cancer treatment based on the change(s) detected in KRAS gene mutation(s). Change(s) in KRAS gene mutation(s) in the subject can determined by detecting one or more mutations in KRAS gene in the subject (1) during the subject is treated for cancer, (2) before the subject is treated for cancer, (3) after the subject is treated for cancer, or a combination thereof. For example, KRAS gene mutation(s) can be detected in the subject immediate before the subject is administered with the PLK1 inhibitor (i.e., pre-dosing), and detected again (once or multiple times) during the first cycle of the cancer treatment. In some embodiments, the KRAS gene mutation(s) is detected at the end of the first cycle of the cancer treatment.

In some embodiments, detecting change(s) in KRAS gene mutation(s) in the subject comprises detecting KRAS gene mutations two or more times in the subject. For example, the KRAS gene mutation(s) can be detected twice, three times, four times, five times, and each time independently at day 1, day 2, day 3, day 5, day 7, day 10, day 11, day 12, day 13, day 14, day 15, day 16, day 17, day 18, day 19, day 20, day 21, day 22, day 23, day 24, day 25, day 26, day 27, day 28, or a number or a range between any two of these values, of the cancer treatment. In some embodiments, the detection of KRAS gene mutation(s) occur at day 5, 7, 14 or 28 of the cancer treatment or the first cycle of the cancer treatment. In some embodiments, the KRAS gene mutation(s) is detected every day during the cancer treatment.

Change(s) in KRAS gene mutation(s) can be, or comprise, change(s) in KRAS gene mutation(s) during the subject is treated for cancer, change(s) in KRAS gene mutation(s) from before the subject is treated for cancer to during the subject is treated for cancer, or a combination thereof. For example, the KRAS gene mutation(s) in the subject can change after the subject starts to receive the cancer treatment (as compared to pre-dosing). In some embodiments, the subject develops different KRAS gene mutation(s) or different variant allele frequency of KRAS during the cancer treatment. The variant allele frequency can be, for example, mutant allelic frequency (MAF). The variant allele frequency of KRAS gene can be determined by total mutation count, mean variant allele frequency, number of KRAS mutation alleles per unit (e.g., ml) of sample (e.g., plasma sample), or both. In some embodiments, the subject has one or more mutations in KRAS gene before being treated with the PLK1 inhibitor. In some embodiments, the subject does not have mutations in KRAS gene before being treated with the PLK1 inhibitor. The KRAS mutations comprises, but are not limited to, G12A, G12C, G12D, G12R, G12S, G12V, G13C, G13D, G13S, G13R, A18D, G61H, Q61L, Q61K, Q61R, K117N, A146T, A146V, A146P, A11V, or a combination thereof.

In some embodiments, detecting change(s) in KRAS gene mutation(s) in the subject comprises detecting change(s) in KRAS gene mutation(s) in a biological sample from the subject, or derivative thereof. The biological sample can be, or comprise, a bodily fluid, whole blood, plasma, one or more tissues, one or more cells, or a combination thereof. The bodily fluid can be, or comprise, blood, plasma, urine, or a combination thereof. The biological sample can comprise ctDNA, CTC, or a combination thereof.

In the methods described herein, determining the responsiveness of the subject comprises determining if the subject is a responder of the treatment, if the subject is or is going to be in complete recover (CR), or if the subject is or is going to be in partial remission (PR). In some embodiments, determining the responsiveness of the subject comprises determining if the subject has a partial response to the treatment, if the subject has a complete response to the treatment, if the subject has a stable disease (SD) status, or if the subject has a progressive disease (PD) status. In some embodiments, the KRAS mutation is measured by determining the amount of the KRAS mutation in proportion to the amount of total KRAS in the sample.

The cancer treatment with the PLK1 inhibitor can be maintained, for example, if the change in MAF of KRAS is a decrease of at least 25%, at least 50%, or at least 75%. Such a decrease can, for example, be detected at the end of cycle 1 of the cancer treatment or at day 1 of cycle 2 of the cancer treatment. In some embodiments, the cancer treatment is maintained if there is at least a 50% decrease in MAF of KRAS. In some embodiments, the cancer treatment is maintained if there is at least a 75% decrease in MAF of KRAS. In some embodiments, the cancer treatment with the PLK1 inhibitor is maintained if KRAS mutation in the samples decreases to below 0.01% or below 0.001% of KRAS in the sample.

The cancer treatment with the PLK1 inhibitor can be modified or discontinued if the change in MAF of KRAS is a decrease of less than 50%, less than 25%, or less than 10%. Such a decrease can, for example, be detected at the end of cycle 1 of the cancer treatment or at day 1 of cycle 2 of the cancer treatment. In some embodiments, the cancer treatment is modified or discontinued if there is less than 50% decrease in MAF of KRAS. In some embodiments, the cancer treatment is modified or discontinued if there is less than 25% decrease in MAF of KRAS. In some embodiments, the cancer treatment with the PLK1 inhibitor is modified or discontinued if KRAS mutation in the samples does not decrease to below 0.01% or below 0.001% of KRAS in the sample. In some embodiments, detecting change(s) in KRAS gene mutation(s) in the subject comprising detecting one or more KRAS mutations emerged in the subject after the subject being treated with the PLK1 inhibitor.

Also disclosed herein includes a method of improving outcome of a cancer treatment. The method comprises: detecting variant allele frequency of KRAS gene in a subject at a first time point in a first sample, wherein the first time point is before the subject starts the cancer treatment, or during the cancer treatment, and wherein the cancer treatment comprises administering a PLK1 inhibitor to the subject; detecting variant allele frequency in KRAS gene in the subject at one or more additional time points in one or more additional samples after the subject, wherein the at least one of the one or more additional time points is during the cancer treatment; determining the difference of the variant allele frequency of KRAS between the first and the one or more additional samples, wherein a decrease in the variant allele frequency in at least one of the one or more additional samples relative to the first sample indicates the subject as responsive to the cancer treatment; and continuing the cancer treatment to the subject if the subject is indicated as responsive to the cancer treatment, or discontinuing the cancer treatment to the subject and/or starting a different cancer treatment to the subject if the subject is not indicated as responsive to the cancer treatment. The first time point can be before the subject starts the cancer treatment. In some embodiments, at least two of the additional time points are during the cancer treatment.

Also disclosed herein includes a method of treating cancer, comprising: treating a subject with cancer, wherein the treating comprises administering a PLK1 inhibitor to the subject; determining a decrease, relative to a variant allele frequency of KRAS gene in a first sample of the subject obtained at a first time point before the subject receives the cancer treatment or during the cancer treatment, in a variant allele frequency of KRAS gene in a second sample of the subject obtained at a second time point after the subject starts receiving the cancer treatment; and continuing with the cancer treatment. In some embodiments, the first time point is prior or immediately prior to the cancer treatment. In some embodiments, the first time point is during the cancer treatment. For example, the first time point is at day 1, day 2, day 3, day 5, day 7, day 10, day 11, day 12, day 13, day 14, day 15, day 16, day 17, day 18, day 19, day 20, day 21, day 22, day 23, day 24, day 25, day 26, day 27, day 28 of the cancer treatment. In some embodiments, the first time point is at day 5, 7, 14, or 28 of the cancer treatment.

In some embodiments, at least one of the one or more additional time points is during the cancer treatment. For example, the first time point is at day 1, day 2, day 3, day 5, day 7, day 10, day 11, day 12, day 13, day 14, day 15, day 16, day 17, day 18, day 19, day 20, day 21, day 22, day 23, day 24, day 25, day 26, day 27, day 28 of the cancer treatment. In some embodiments, at least one of the one or more additional time points is at day 5, 7, 14, or 28 of the cancer treatment. In some embodiments, the one or more additional time points is during the cancer treatment. For example, the first time point is at day 1, day 2, day 3, day 5, day 7, day 10, day 11, day 12, day 13, day 14, day 15, day 16, day 17, day 18, day 19, day 20, day 21, day 22, day 23, day 24, day 25, day 26, day 27, day 28 of the cancer treatment. In some embodiments, the one or more additional time points is at day 5, 7, 14, or 28 of the cancer treatment. In some embodiments, the first time point and at least one of the one or more additional time points are during the first cycle of the cancer treatment. In some embodiments, at least one of the one or more additional time points are during the first cycle of the cancer treatment, and at least one of the one or more additional time points are during the second cycle of the cancer treatment.

As disclosed herein, the variant allele frequency is, in some embodiments, mutant allelic frequency (MAF). The variant allele frequency of KRAS gene can be determined by, for example, total mutation count, mean variant allele frequency, number of KRAS mutation alleles per unit (e.g., ml) of the sample (e.g., plasma sample), or any combination thereof. In some embodiments, detecting variant allele frequency in KRAS gene comprises detecting variant allele frequency in KRAS gene in a biological sample from the subject, or derivative thereof. The biological sample can be, or comprise, a bodily fluid, whole blood, plasma, one or more tissues, one or more cells, or a combination thereof. In some embodiments, the bodily fluid comprises blood, plasma, urine, or a combination thereof. In some embodiments, the biological sample comprises circulating tumor DNA (ctDNA), circulating tumor cell (CTC), or a combination thereof. In some embodiments, droplet digital PCR (ddPCR), polymerase chain reaction (PCR) or next generation sequencing (NGS) is used to determine MAF of KRAS.

The subject can have one or more mutations in KRAS gene before being treated with the PLK1 inhibitor. In some embodiments, the subject does not have mutations in KRAS gene before being treated with the PLK1 inhibitor. In some embodiments, the subject has received one or more prior cancer treatment. The cancer can be advanced, metastatic, refractory, or relapsed. The cancer can be colorectal cancer (e.g., metastatic colorectal cancer), pancreatic cancer, leukemia, lung cancer, or a combination thereof.

The KRAS gene mutation(s) can be, or comprise, mutations at codon 12, codon 13, codon 18, codon 61, codon 117, codon 146, or a combination thereof. In some embodiments, the KRAS gene mutation(s) comprise mutations at codon 12 and/or codon 13. Non-limiting examples of the KRAS gene mutation(s) include G12A, G12C, G12D, G12R, G12S, G12V, G13C, G13D, G13S, G13R, A18D, G61H, Q61L, Q61K, Q61R, K117N, A146T, A146V, A146P, A11V, or a combination thereof.

In some embodiments, the PLK1 inhibitor is onvansertib. In some embodiments, the treatment comprises administration of onvansertib for ten days in a cycle of 28 days. For example, the treatment can comprise administration of onvansertib for five days in the first 14 days and five days in the second 14 days in a cycle of 28 days. In some embodiments, the cancer treatment comprises administering to the subject at least one additional cancer therapeutics or cancer therapy, including but not limited to, FOLFIRI, bevacizumab, abiraterone, or a combination thereof. In some embodiments, the PLK inhibitor and the cancer therapeutics or cancer therapy are co-administered simultaneously or sequentially.

In some embodiments, the cancer treatment comprises one or more cycles, and change(s) in KRAS gene mutation(s) or variant allele frequency of KRAS is detected before, during and/or after each cycle of the leukemia treatment. Each cycle of treatment can be at least 21 days, for example, from about 21 days to about 28 days.

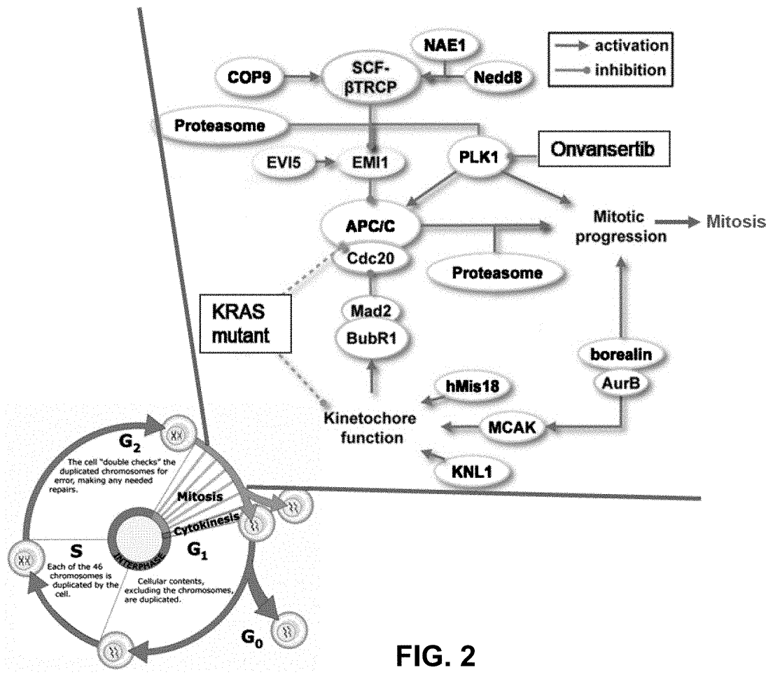

As disclosed herein, periodic measurement of one or more KRAS mutations in ctDNA, for example in a bodily fluid, of a cancer patient can provide early indication of the effectiveness of treatment being administered to the patient. Examples provided herein showed that where PLK1 inhibition by onvansertib is an effective treatment, in combination with standard-of-care FORFIRI and bevacizumab, for metastatic colorectal cancer having a KRAS mutation. Without being bound to any particular theory, it is believed that KRAS mutants are experiencing mitotic stress and exacerbating this stress in particular ways such that interference with PLK1 leads to stress overload and tumor cell death. As shown in FIG. 2, both KRAS mutants and PLK1 inhibition block the anaphase promoting complex (APC/C), which is crucial for mitosis to occur. The APC/C complex is critical in mediating the metaphase to anaphase transition. The dual block of APC/C from PLK1 inhibitor treatment is believed to cause the synthetic lethality to KRAS mutant cancer cells, as illustrated in FIG. 1.

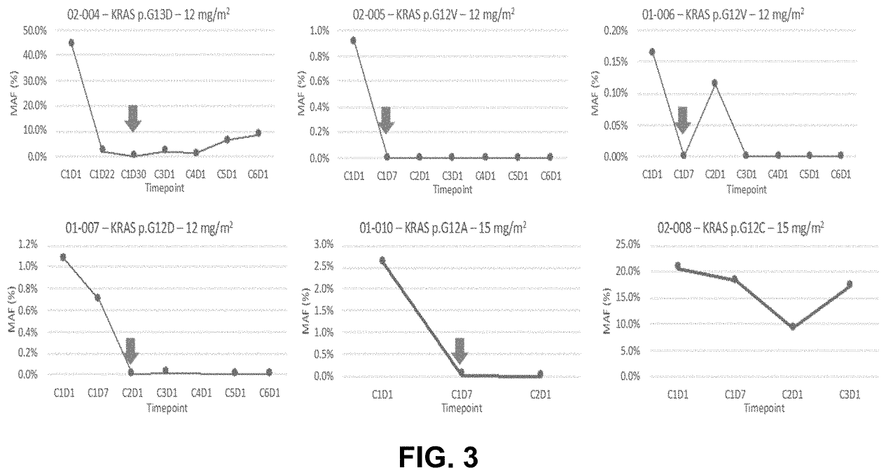

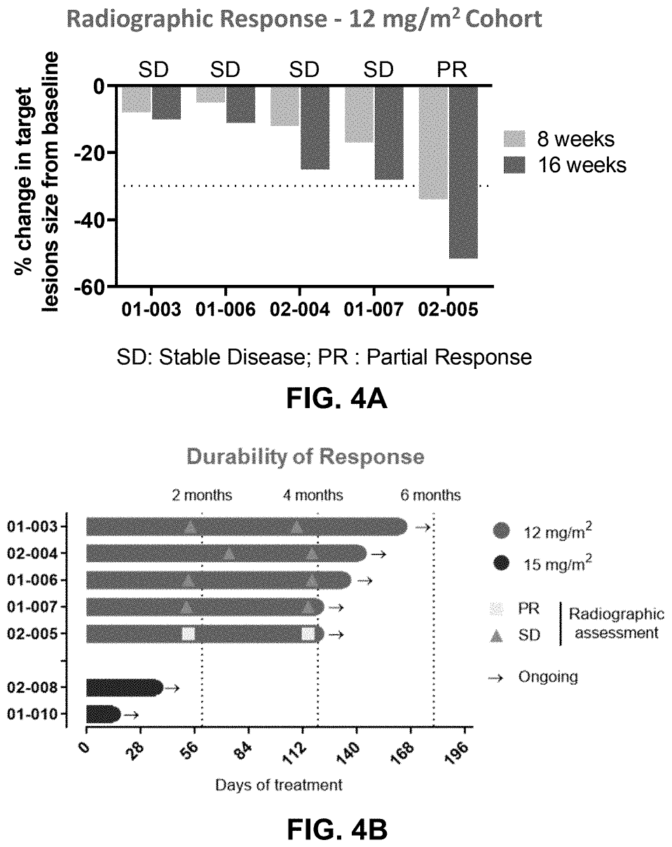

The combination treatment caused a reduction of mutant KRAS in plasma cell-free DNA to below the level of detection (0.001% mutant KRAS) within a month (in some cases within a week) of the start of the treatment (FIG. 3). This reduction correlated with radiographic response in those patients (FIG. 4A). Those findings establish that periodic measuring KRAS mutations in ctDNA of cancer patients is useful for quickly determining treatment effectiveness.

Provided herein include a method comprising: (a) treating a patient for a cancer characterized by a KRAS mutation, and (b) periodically sampling a bodily fluid from the patient and measuring the KRAS mutation in cell-free DNA in the bodily fluid.

These methods are useful for evaluating a treatment for any cancer characterized by a KRAS mutation. Nonlimiting examples include leukemia, lung cancer, colorectal cancer, and pancreatic cancer. In some embodiments, the cancer is colorectal cancer. In some of those embodiments, the cancer is metastatic colorectal cancer. In some embodiments, the cancer is a cancer associated with one or more KRAS mutations.

Any treatment of a cancer characterized by a KRAS mutation can be evaluated using these methods. Non-limiting examples include surgery, chemotherapy, radiation therapy (including external-beam, stereotactic, and intraoperative radiation therapy and brachytherapy), bone marrow transplant, immunotherapy, targeted drug therapy, cryoablation, or radiofrequency ablation. Where the cancer is colorectal cancer, examples of treatments include surgery, radiofrequency ablation, cryoablation, radiation therapy, chemotherapy (including medications comprising capecitabine, 5-FU, irinotecan, oxaliplatin, trifluridine/tipiracil), targeted therapy (including anti-angiogenesis therapy using, for example bevacizumab, regorafenib, ziv-aflibercept, or ramucirumab; immunotherapy using, for example pembrolizumab, nivolumab, or ipilimumab; and PLK1 inhibitors).

In some embodiments, the treatment comprises administration of a PLK1 inhibitor. Nonlimiting examples include onvansertib, BI2536, volasertib (BI 6727), GSK461364, HMN-176, HMN-214, AZD1775, CYC140, rigosertib (ON-01910), MLN0905, TKM-080301, TAK-960 or Ro3280. In various embodiments, the PLK1 inhibitor is onvansertib.

Any bodily fluid that would be expected to have nucleic acids can be utilized in these methods. Non-limiting examples of bodily fluids include peripheral blood, serum, plasma, urine, lymph fluid, amniotic fluid, and cerebrospinal fluid. In various embodiments the bodily fluid is blood, plasma or urine.

The method can be applied to a patient having any cancer having KRAS mutation now known or later discovered. The KRAS mutation can be G12D, G12V, G13D, G12C, G12S, G12A, or G12R. In some embodiments, the KRAS mutation is A18D, Q61H, or K117N.

As shown in Example 1 provided herein, describing treatment of mCRC with onvansertib, FOLFIRI and bevacizumab, the KRAS mutant can become nondetectable (less than 0.001% of KRAS) within one week of the start of treatment. In these methods, the samples can be taken at any time in relation to the beginning of the treatment. In some embodiments, a sample is taken prior to, or at, the beginning of the treatment. In some embodiments, a sample is taken more than once after the start of treatment, e.g., at least twice within one after administration of the treatment. In some embodiments, a sample is taken within one week after the beginning of treatment. In some embodiments, at least two samples of the bodily fluid are taken within one month of starting the treatment. In further embodiments, a sample is taken within one month from the beginning the treatment. As shown in the Examples herein, describing treatment of mCRC with onvansertib, FOLFIRI and bevacizumab, the KRAS mutant can become nondetectable (less than 0.001% of KRAS) within one week of the start of treatment.

The KRAS mutation in the sample can be measured by any method now known or later discovered. Nonlimiting examples include any PCR and any next-generation sequencing (NGS) method. In some embodiments, the method is droplet digital PCR. With those methods, any parameter of the KRAS mutant in the sample can be measured, for example total KRAS mutant in a specific volume of bodily fluid, or amount of the KRAS mutation in proportion to the amount of total KRAS in the sample (as in Example).

The methods, composition and kits disclosed herein can be utilized to make treatment decisions, e.g., whether to maintain the treatment or modify the treatment. The particular level of KRAS mutant in the sample that directs the treatment recommendation can be determined for any particular treatment and cancer without undue experimentation, optionally taking into consideration any other particular factors that could affect the recommendation, e.g., possible interactions with other medications taken by the patient, other patient disorders that could affect the effectiveness or tolerance of the treatment, etc.

In some embodiments of these methods, (i) if the KRAS mutation in the samples decreases to below a set percentage of KRAS in the sample, the treatment is maintained, or (ii) if the KRAS mutation in the samples does not decrease to below the set percentage of KRAS in the sample, the treatment is modified. In these embodiments, the percentage of the KRAS in the sample that is the threshold between maintaining and modifying the treatment can be determined by consideration of any number of factors, for example the sensitivity of the assay and past results with other patients. In various embodiments, the percentage decrease, above which treatment modification is indicated, is 0.1%, 0.05%, 0.01%, 0.005%, 0.001%, or any percentage in between or outside those percentages. In some embodiments, the percentage is 0.01%; in other embodiments the percentage is 0.001%.

When the method indicates that the treatment should be modified, the modified treatment can comprise any of the treatments discussed above.

In some embodiments of these methods, the KRAS mutation emerged in a resistant tumor of the cancer after the patient was treated for the cancer having wild-type KRAS. In some of these embodiments, the cancer is mCRC.

### Kits

Disclosed herein include kits for determining responsiveness of a subject to a cancer treatment, kits for improving outcome of a cancer treatment, and kits for treating cancer. The kit, in some embodiments, comprises: a PLK1 inhibitor (e.g., onvansertib) or a pharmaceutically acceptable salt, solvate, stereoisomer thereof, and a manual providing instructions for performing one or more of the steps for one or more methods disclosed herein.

In some embodiments, the kit comprises: a PLK1 inhibitor (e.g., onvansertib) or a pharmaceutically acceptable salt, solvate, stereoisomer thereof, and manual providing instructions for performing one or more steps of the method disclosed herein for determining responsiveness of a subject to a cancer treatment. For example, the method can comprise: treating a subject with cancer, wherein the treating comprises administering a PLK1 inhibitor to the subject; detecting change(s) in KRAS gene mutation(s) in the subject, and determining the responsiveness of the subject to the cancer treatment based on the change(s) detected in KRAS gene mutation(s). In some embodiments, detecting change(s) in KRAS gene mutation(s) in the subject comprises detecting one or more mutations in KRAS gene in the subject (1) during the subject is treated for cancer, (2) before the subject is treated for cancer, (3) after the subject is treated for cancer, or a combination thereof. Detecting change(s) in KRAS gene mutation(s) can comprise detecting variant allele frequency of KRAS gene, for example MAF of KRAS gene. The cancer treatment with the PLK1 inhibitor can be maintained if the change in MAF of KRAS is a decrease of at least 25%, at least 50%, or at least 75%. In some embodiments, the decrease is detected at the end of cycle 1 of the cancer treatment or at day 1 of cycle 2 of the cancer treatment. The cancer treatment with the PLK1 inhibitor can be, for example, modified or discontinued if the change in MAF of KRAS is a decrease of less than 50%, less than 25%, or less than 10%. In some embodiments, the decrease is detected at the end of cycle 1 of the cancer treatment or at day 1 of cycle 2 of the cancer treatment.

In some embodiments, the kit comprises: a PLK1 inhibitor (e.g., onvansertib) or a pharmaceutically acceptable salt, solvate, stereoisomer thereof, and manual providing instructions for performing one or more steps of the method disclosed herein for improving outcome of a cancer treatment. For example, the method can comprise: detecting variant allele frequency of KRAS gene in a subject at a first time point in a first sample, wherein the first time point is before the subject starts the cancer treatment, or during the cancer treatment, and wherein the cancer treatment comprises administering a PLK1 inhibitor to the subject; detecting variant allele frequency in KRAS gene in the subject at one or more additional time points in one or more additional samples after the subject, wherein the at least one of the one or more additional time points is during the cancer treatment; determining the difference of the variant allele frequency of KRAS between the first and the one or more additional samples, wherein a decrease in the variant allele frequency in at least one of the one or more additional samples relative to the first sample indicates the subject as responsive to the cancer treatment; and continuing the cancer treatment to the subject if the subject is indicated as responsive to the cancer treatment, or discontinuing the cancer treatment to the subject and/or starting a different cancer treatment to the subject if the subject is not indicated as responsive to the cancer treatment. The first time point can be, for example, before the subject starts the cancer treatment. In some embodiments, at least two of the additional time points are during the cancer treatment. In some embodiments, the kit comprises: a PLK1 inhibitor (e.g., onvansertib) or a pharmaceutically acceptable salt, solvate, stereoisomer thereof, and manual providing instructions for performing one or more steps of the method disclosed herein for treating cancer. For example, the method can comprise: treating a subject with cancer, wherein the treating comprises administering a PLK1 inhibitor to the subject; determining a decrease, relative to a variant allele frequency of KRAS gene in a first sample of the subject obtained at a first time point before the subject receives the cancer treatment or during the cancer treatment, in a variant allele frequency of KRAS gene in a second sample of the subject obtained at a second time point after the subject starts receiving the cancer treatment; and continuing with the cancer treatment. Detecting variant allele frequency of KRAS gene can be, for example, detecting MAF of KRAS gene. The variant allele frequency of KRAS gene can be determined by total mutation count, mean variant allele frequency, number of KRAS mutation alleles per unit (e.g., ml) of sample (e.g., plasma sample), or any combination thereof.

The KRAS gene mutations can comprise mutations at codon 12, codon 13, codon 18, codon 61, codon 117, codon 146, or a combination thereof, for example mutations at codon 12 and/or codon 13. Non-limiting examples of KRAS gene mutation include G12A, G12C, G12D, G12R, G12S, G12V, G13C, G13D, G13S, G13R, A18D, G61H, Q61L, Q61K, Q61R, K117N, A146T, A146V, A146P, A11V, or a combination thereof.

The instructions can comprise instructions for administering the PLK1 inhibitor at 9 mg/m²-90 mg/m², for example at from 9 mg/m² to 24 mg/m². For example, the instructions can be for administering the PLK1 inhibitor at 12 mg/m², 15 mg/m², or 18 mg/m².

Some embodiments provided herein provide a method comprising (a) treating a patient for a cancer characterized by a KRAS mutation, and (b) periodically sampling a bodily fluid from the patient and measuring the KRAS mutation in cell-free DNA in the bodily fluid. The cancer can be leukemia, lung cancer, colorectal cancer (e.g., metastatic colorectal cancer), or pancreatic cancer. In some embodiments, the treatment comprises administration of a polo-like kinase 1 (PLK1) inhibitor, including but are not limited to one or more of onvansertib, BI2536, volasertib (BI 6727), GSK461364, HMN-176, HMN-214, AZD1775, CYC140, rigosertib (ON-01910), MLN0905, TKM-080301, TAK-960, and Ro3280. In some embodiments, the PLK1 inhibitor is onvansertib. The bodily fluid can be blood, plasma or urine. In some embodiments, the KRAS mutation is G12D, G12V, G13D, G12C, G12S, G12A, or G12R. In some embodiments, the KRAS mutation is G13D, G12V, G12D, G12A or G12C. The KRAS mutation can be measured, for example, in at least two samples of the bodily fluid that are taken within one month of starting the treatment. The KRAS mutation can be measured, for example, by determining the amount of the KRAS mutation in proportion to the amount of total KRAS in the sample.

The treatment can be modified, in some embodiments, (i) if the KRAS mutation in the samples decreases to below 0.01% of KRAS in the sample, the treatment is maintained, or (ii) if the KRAS mutation in the samples does not decrease to below 0.01% of KRAS in the sample, or both (i) and (ii). The treatment can be maintained, in some embodiments, (i) if the KRAS mutation in the samples decreases to below 0.001% of KRAS in the sample, the treatment is maintained, or (ii) if the KRAS mutation in the samples does not decrease to below 0.001% of KRAS in sample, or both (i) and (ii). In some embodiments, the decrease in the KRAS mutation is determined on samples taken within one month of starting the treatment. In some embodiments, the KRAS mutation emerged in a resistant tumor of the cancer after the patient was treated for the cancer having wild-type KRAS. The cancer can be metastatic colorectal cancer.

Also provide includes a method comprising (a) treating a patient for colorectal cancer (e.g., metastatic colorectal cancer) characterized by a KRAS mutation, and (b) periodically sampling a bodily fluid from the patient and measuring the KRAS mutation in cell-free DNA in the bodily fluid, wherein the treatment comprises administration of a PLK1 inhibitor, for example onvansertib. In some embodiments, the KRAS mutation emerged in a resistant tumor of the cancer after the patient was treated for the cancer having wild-type KRAS.

### EXAMPLES

Some aspects of the embodiments discussed above are disclosed in further detail in the following example, which are not in any way intended to limit the scope of the present disclosure.

### Example 1

### Measurement of KRAS Mutations in Cell-Free DNA of Metastatic Colorectal Cancer Patients

The clinical trial NCT03829410, Onvansertib in Combination With FOLFIRI and Bevacizumab for Second Line Treatment of Metastatic Colorectal Cancer Patients With a Kras Mutation, is a Phase 1b/2 study to determine the safety and efficacy of Onvansertib, administered orally, daily, for 5 consecutive days on Day 1-5 of each 14-day course in each 28-day cycle, in combination with FOLFIRI+Avastin, as second-line treatment in adult patients who have metastatic colorectal cancer with a KRAS mutation. Participants must have histologically confirmed metastatic and unresectable disease, and previously failed treatment or be intolerant to fluoropyrimidine and oxaliplatin with or without bevacizumab.

KRAS in cell free DNA in plasma from patients was periodically quantified using droplet digital PCR (BioRad), where percentage of KRAS that is mutant KRAS was determined. The lower limit of sensitivity of this assay was 0.001% mutant KRAS.

Five different KRAS mutant variants were detected in 6 patients, which represents >90% of KRAS mutations in CRC; all five KRAS variants decreased within the first cycle of treatment (onvansertib dose levels 12 and 15 mg/m²).

At dose level 1 (onvansertib 12 mg/m²), 4 patients had detectable KRAS mutant ctDNA at baseline; in all 4 patients KRAS was undetectable within the 1st cycle of treatment; this preceded subsequent tumor shrinkage observed with radiographic scans, supporting the predictive value of the invention methods.

At dose level 2 (onvansertib 15 mg/m²), the 2 patients treated to-date had detectable KRAS mutant ctDNA at baseline; in 1 patient KRAS was undetectable within the 1st cycle of treatment.

Decreases in plasma KRAS mutation level has been asserted to be an early marker for therapeutic response (confirmed by subsequent radiographic scans) (Tie et al., 2015). However, although the “no response” group had higher average KRAS levels than the group showing a response, that difference was not statistically significant (Table 2 of Tie et al.). The data provided here shows a much greater reduction in mutant KRAS among responders, to undetectable levels (less than 0.001%) whereas the responders in Tie et al. showed a minimum mutant KRAS of 0.2%, while samples in the “no response” group went as low as 0.3%. Nonetheless, Tie et al. do show that a less than 10-fold reduction in mutant KRAS in plasma correlates with “no response” whereas a greater than 10-fold reduction correlates with positive response to treatment.

### Example 2

### KRAS Mutations in Metastatic Colorectal Cancer Patients

A Phase 1b/2 Study of onvansertib, in combination with FOLFIRI and Bevacizumab for second-line treatment of patients with KRAS-mutated metastatic colorectal cancer (mCRC) was conducted.

Safe and effective second-line treatment is needed in KRAS-mutated metastatic colorectal cancer (mCRC). KRAS is mutated in 50% of CRC patients and to-date, RAS-therapies have failed with the majority of KRAS mutations considered to be undruggable: Anti-farnesyl inhibitors and inhibitors of downstream effectors of RAS show no, or limited, efficacy, and covalent inhibitors of KRAS G12C (representing 8% of KRAS mutations in CRC) have shown limited activity in CRC. Second-line treatments (chemotherapy±targeted agents) has poor prognosis: ORR being about 5%, PFS being about 5.7 months, OS being about 11.2 months. Third- or later-lines of treatment have poor outcomes: approved therapies for KRAS mutant patients are regorafenib and trifluridine/tipiracil (TAS-102) which have a median progression-free survival (PFS) of 2-3 months and median overall survival of 6-9 months. In addition, alternative strategies to inhibit KRAS include targeting synthetic lethal partners of mutant KRAS (i.e., proteins that are essential in KRAS-mutant but not wild-type cells) are needed.

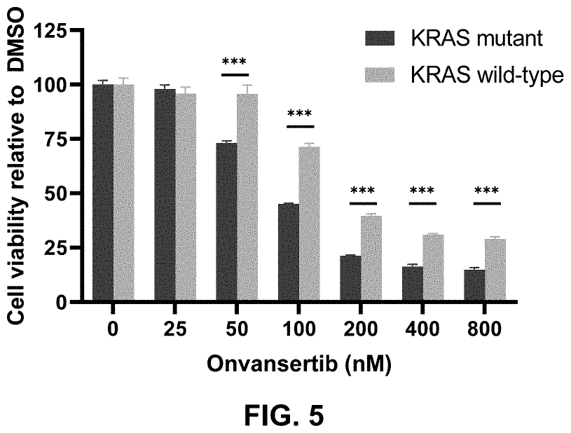

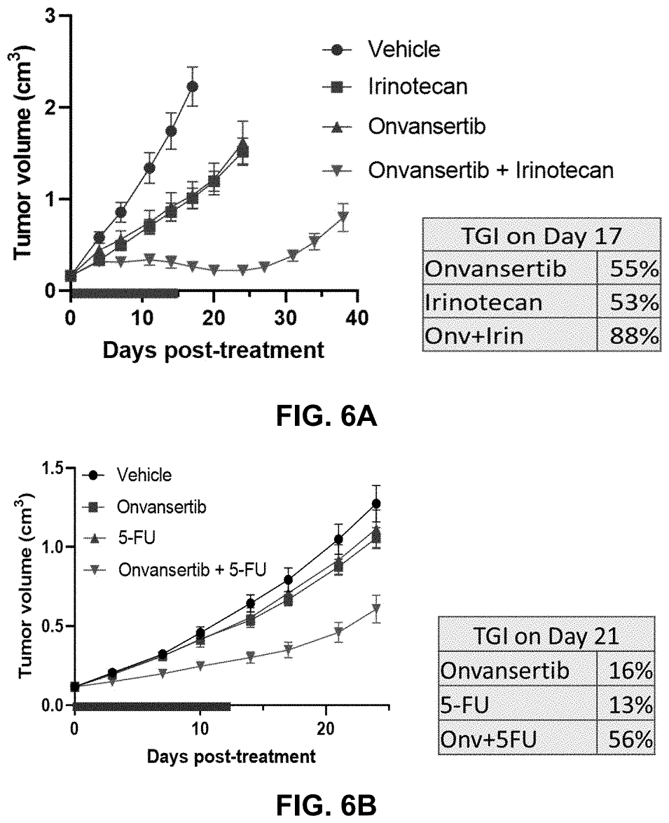

Onvansertib, an oral and highly-selective PLK1 inhibitor, is a promising therapeutic option for KRAS-mutated CRC. PLK1, a key regulator of mitosis, is overexpressed in CRC and associated with poor clinical parameters. A genome-wide RNAi screen identified PLK1 inhibition to be synthetic lethal with mutant KRAS in CRC cells, and showed that KRAS mutant cells were hypersensitive to inhibition of PLK1, and onvansertib induced more profound mitotic arrest and cell death in mutant KRAS cells than wild-type (WT) cells (FIG. 5 shows cell viability in onvansertib-treated KRAS mutant and WT isogenic CRC cells. In FIG. 5, DLD-1 cells were treated for 72h with DMSO or onvansertib. Cell viability was measured with CellTiterGlo. Two-way ANOVA, p<0.0001). In addition, as shown in FIGS. 6A-6B, onvansertib induced potent anti-tumor activity as single agent and showed synergy in combination with irinotecan and 5-FU in the HCT-116 KRAS mutant xenograft model. In FIGS. 6A and 6B, the thick bar represent treatment, data a represented as mean tumor volume±SEM, and TGI refers to tumor growth inhibition.

### Phase 1b/2 Study

Study Design and Objectives

Key Eligibility criteria: (1) Metastatic and unresectable CRC, (2) KRAS mutation in primary tumor or metastasis, (3) Has failed treatment or is intolerant to oxaliplatin-based chemotherapy, (4) Progression <6 months of first-line or maintenance therapy, and (5) Negative for BRAF V600E mutation and MSI-H/dMMR.

Study design: Phase 1b: onvansertib dose escalation (12, 15, 18 mg/m²) in successive cohorts of 3 patients and dose limiting toxicities (DLTs) evaluated during the 1st cycle (28 days). Phase 2: expansion cohort t the MTD or RP2D.

Efficacy endpoints: (1) Primary: objective response rate (ORR) in patients who receive at least 1 cycle of treatment, and (2) secondary: Progression-free survival (PFS) and reduction in KRAS allelic burden assessed by liquid biopsies.

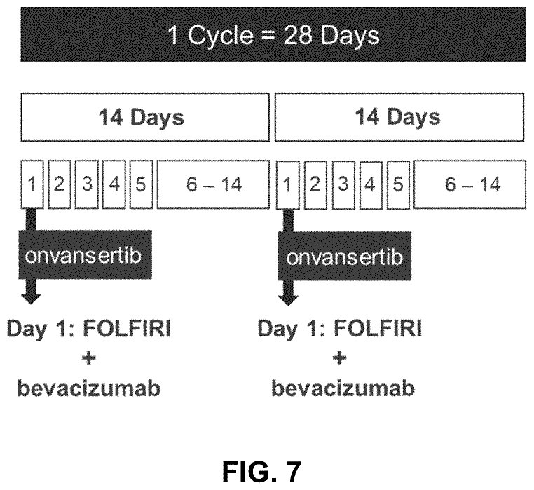

Treatment schedule is shown in FIG. 7.

### Results

Total of 12 patients were enrolled in the study as of May 4, 2020 (Table 1). Safety was demonstrated with the combination of onvansertib+FOLFIRI/bevacizumab. Onvansertib 12 and 15 mg/m² dose levels were cleared for safety; Onvansertib 18 mg/m²: DLT-G4 neutropenia in 1 of 3 patients, deemed to be associated with 5FU bolus; enrolling 3 additional patients. All grade 3-4 adverse events resolved within 2.5 weeks and did not result in treatment discontinuation.

TABLE 1
Patient enrollment

| | Cohort 1 | Cohort 2 | Cohort 3 |
|---|---|---|---|
| | Onvansertib | Onvansertib | Onvansertib |
| Number of patients (N) | 12 mg/m² | 15 mg/m² | 18 mg/m² |
| Treated | 6 | 3 | 3 |
| Completing 1^{st} cycle | 6 | 3 | 3 |
| Currently on Treatment | 3 | 2 | 3 |

Efficacy:

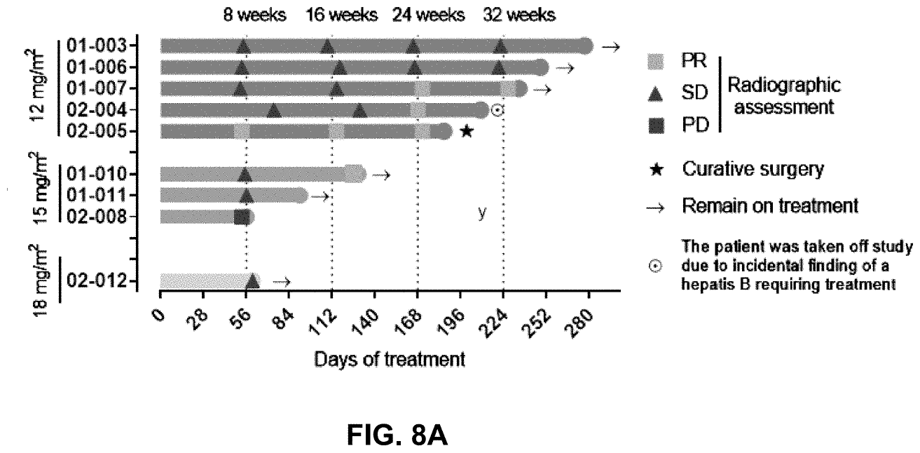

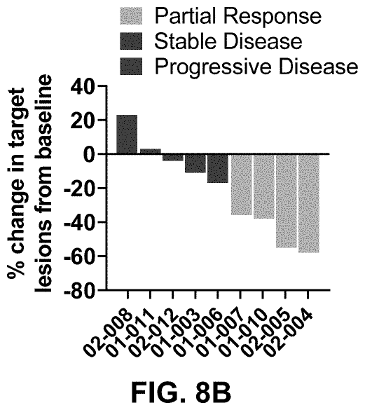

Preliminary efficacy was demonstrated with onvansertib+FOLFIRI/bevacizumab: Of the 9 evaluable patients, 8 (89%) had clinical benefit: 4 (44%) partial response (PR) and 4 (44%) stable disease (SD); 2 patients had confirmed PR; 1 patient (02-005) proceeded to successful curative surgery; Responses appear durable: (1) Median PFS of >6 months to-date, and (2) 6 patients remain on treatment. FIG. 8A shows the results of treatment response and duration, and FIG. 8B shows the results of radiographic response.

Biomarker Analysis:

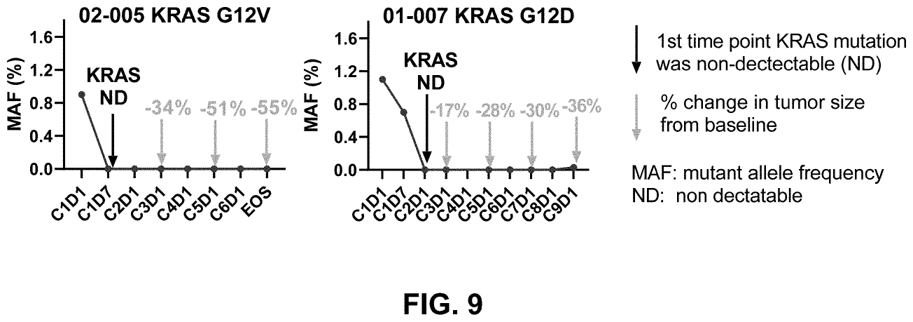

Changes in plasma KRAS mutant during 1st cycle of treatment were found to be highly predictive of tumor regression (FIG. 9). 8 of the 9 patients had a KRAS mutation detected by ctDNA analysis at baseline (detected using ddPCR and NGS). 5 patients had a decrease in KRAS mutant to a non-detectable level in cycle 1 and subsequent tumor regression at 8 weeks (C3D1).

Total of 15 patients were enrolled in the study as of Nov. 4, 2020 (Table 2). 3+3 dose escalation design to assess the safety of the combination and identify the maximum tolerated dose (MTD) and recommended Phase 2 dose (RP2D) of onvansertib.

TABLE 2
Patient enrollment

| | Cohort 1 | Cohort 2 | Cohort 3 |
|---|---|---|---|
| | Onvansertib | Onvansertib | Onvansertib |
| Number of patients (N) | 12 mg/m² | 15 mg/m² | 18 mg/m² |
| Treated | 6 | 3 | 6 |
| Completing 1^{st} cycle | 6 | 3 | 4 |
| Currently on Treatment | 1 | 0 | 2 |

Phase 1b Safety Assessment:

Safety has been demonstrated with the combination of onvansertib+FOLFIRI/bevacizumab, which was well-tolerated. The most common treatment-emergent adverse events (AEs) are shown in Table 3. No major or unexpected toxicities were attributed to onvansertib. Four patients had DLTs attributed to the 5-FU bolus: one G4 neutropenic fever at dose level 12 mg/m²; Three G4 neutropenia at dose level 18 mg/m²; and Dose level 18 mg/m2 exceeded the MTD. 3 additional patients are being enrolled at 15 mg/m² to further explore safety at this dose level. Combination treatment was well tolerated: (1) of all AEs only 9% (17/192) were G3/G4; (2) the only G3/G4 AEs reported in ≥2 patients were neutropenia (n=8); which were managed by dose delay, growth factor support and/or discontinuation of the 5-FU bolus; no patients went off trial due to neutropenia.

TABLE 3
Most common treatment-emergent AEs

| | | | | | All |
|---|---|---|---|---|---|
| Adverse Events (AEs) | Grade 1 | Grade 2 | Grade 3 | Grade 4 | Grades |
| Neutropenia | 1 | 2 | 4 | 4 | 11 |
| Fatigue | 3 | 7 | 1 | | 11 |
| Nausea | 5 | 3 | 1 | | 9 |
| Diarrhoea | 6 | 2 | | | 8 |
| Alopecia | 6 | 1 | | | 7 |
| Abdominal pain | 1 | 4 | 1 | | 6 |
| Anaemia | 4 | 1 | | | 5 |
| WBC decrease | 2 | 3 | | | 5 |
| Vomiting | 2 | 1 | 1 | | 4 |
| Thrombocytopenia | 2 | 2 | | | 4 |
| Mucosal inflammation | 1 | 2 | | | 3 |
| Dyspepsia | 3 | | | | 3 |
| Stomatitis | 2 | 1 | | | 3 |
| ALT increased | 2 | 1 | | | 3 |
| Abdominal distension | 3 | | | | 3 |
| Back pain | 3 | | | | 3 |
| Epistaxis | 3 | | | | 3 |

N = number of patients (total N = 15);
WBC = white blood cells;
ALT = alanine aminotransferase

Phase 1b Preliminary Efficacy:

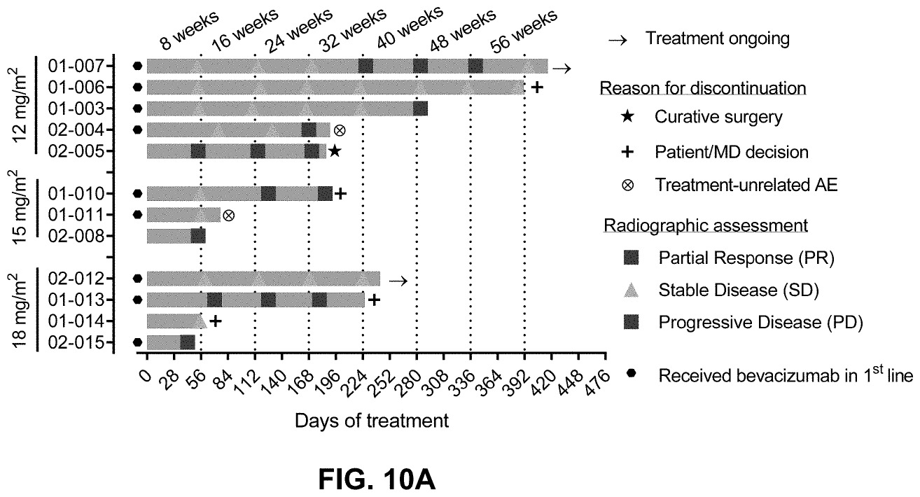

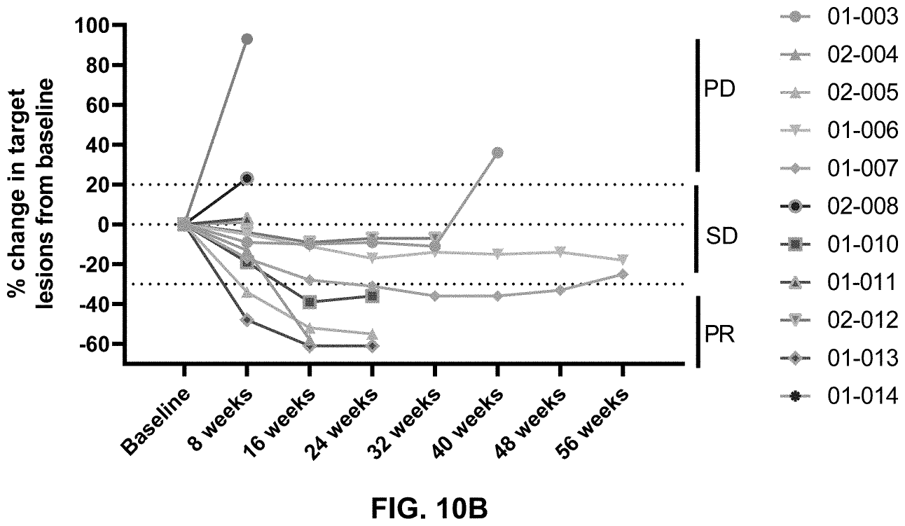

Of the 12 patients evaluable for efficacy (these patients completed 8 weeks of treatment and had radiographic scan or progressed within 8 weeks while on treatment), 5 (42%) patients achieved a partial response (PR) which included 4 patients had a confirmed PR; 1 patient went on to have curative surgery; and 1 patient with non-confirmed PR went off study following PR due to treatment-unrelated AE. In addition, 8 (67%) patients had durable responses of >6 months (range 6.1 to 13.7 months as of data cutoff date). FIG. 10A shows treatment response and duration; and FIG. 10B shows changes in tumor size from baseline.

KRAS Mutant Allelic Frequency (MAF) Biomarker Analyses:

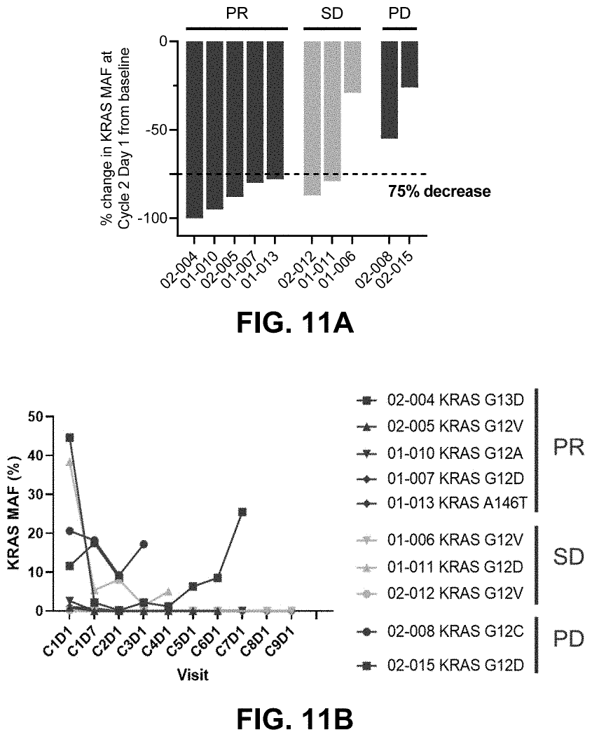

KRAS MAF was measured by digital droplet PCR (ddPCR) at baseline (Cycle 1 Day 1, pre-dose) and on-treatment (Day 1 of Cycles 2 to 9). 10 of 12 patients had a KRAS variant detected by ddPCR at baseline (all had a KRAS mutation detected by NGS). Clinical responses were observed across different KRAS variants, including the 3 most common in CRC. Patients achieving a PR showed the greatest decreases in plasma mutant KRAS after one cycle of therapy. The greatest changes in KRAS MAF after 1 cycle of treatment were observed in patients achieving a PR (ranging from −78% to −100%) while the 2 patients who progressed showed a more modest reduction in KRAS MAF (−55% and −26%). In addition, patients with PR and SD tended to have lower on-treatment KRAS MAF than patients with early PD. FIG. 11A shows % KRAS MAF changes after 1 cycle, and FIG. 11B shows KRAS MAF changes over time.

### Expanded Access Program (EAP)

Key Eligibility criteria: (1) metastatic and unresectable CRC with a confirmed KRAS mutation, (2) participants had failed or progressed on multiple lines of standard-of-care systemic therapy, including prior FOLFIRI, and (3) participant was not eligible for clinical trial.

Treatment: participants received onvansertib (15 mg/m²)+FOLFIRI+Bevacizumab, with an option to eliminate 5-FU bolus. Treatment schedule was as shown in FIG. 7. As of Mar. 10, 2021, 43 participants were enrolled and treated at 22 EAP sites.

Safety: onvansertib in combination with FOLFIRI+Bevacizumab has been well-tolerated with no serious adverse events (SAEs) reported to-date in any of the treated participants (N=43).

Clinical benefit accessed in evaluable EAP participants: as of Mar. 10, 2021, 20 participants had at least one on-treatment radiographic scan and were evaluated for clinical benefit. All participants had median number of 3 prior lines of treatment, and 70% were progressing prior to enrolling in EAP. Participants had a median PFS on EAP of 5.6 months (95% CI: 2.7-NR) and 11 remained on treatment. Baseline characteristics of the participants are shown in Table 4.

TABLE 4
Baseline characteristics of participants

| Baseline Characteristics (n = 20)* | Median [range] or N (%) | |
|---|---|---|
| Participants Age | 50 | [35-74] |
| Prior lines of therapy | 3 | [1-6] |
| Participants who received irinotecan- | 15 | (75%) |
| based regimen as last therapy | | |
| Participants progressing prior to the EAP | 14 | (70%) |

*Participants who had at least one on-treatment radiographic scan

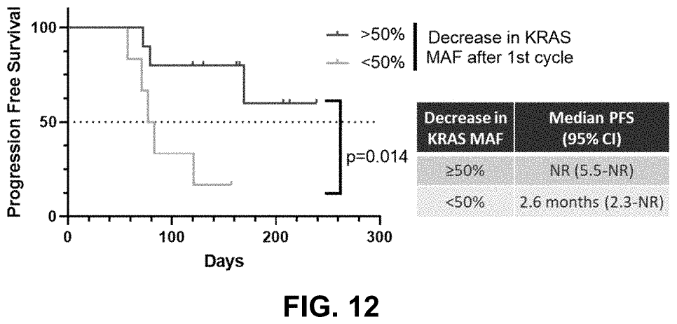

Changes in plasma KRAS-mutant were found to correlate with clinical benefit. KRAS mutant allelic frequency (MAF) was measured by digital droplet PCR (ddPCR) at baseline and end of Cycle 1. 16 of 20 participants had a KRAS variant detected by ddPCR at baseline. Participants with greater than 50% decrease in KRAS MAF (n=10) had a significant increase in PFS compared with participants who had less than 50% decrease (n=6), supporting that early changes in KRAS MAF are predictive of clinical benefit. FIG. 12 shows PFS of participants with detectable plasma KRAS mutant at baseline.

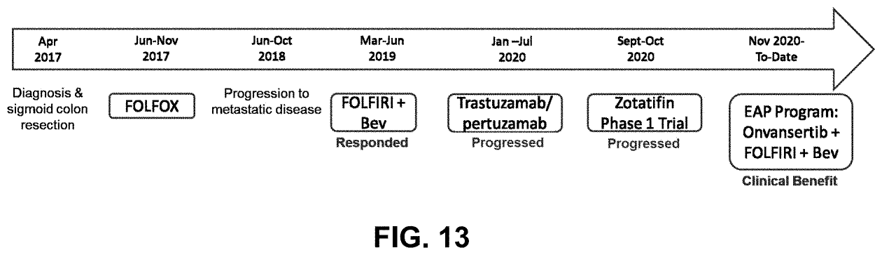

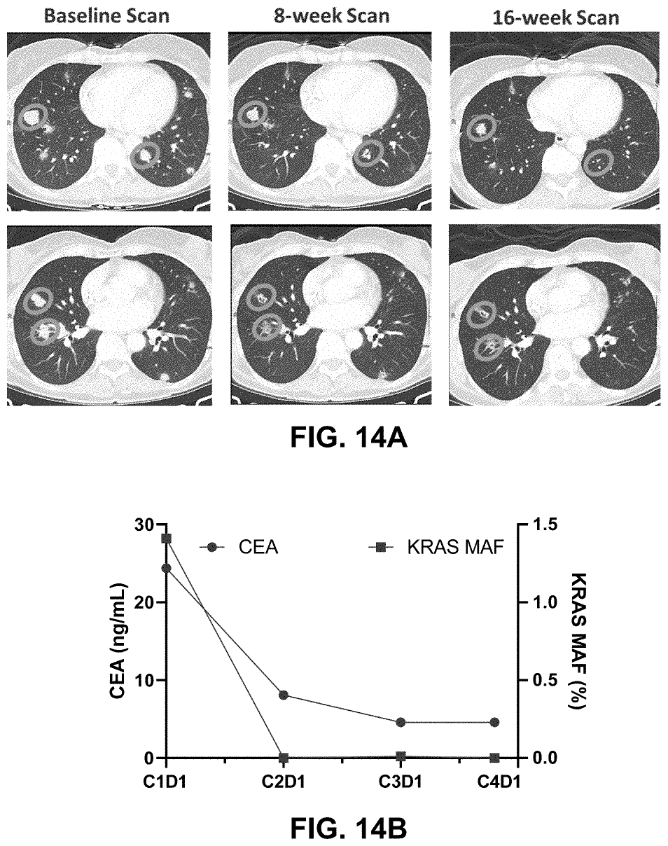

A 61-year-old female with KRAS G12V metastatic sigmoid colon cancer participated in the EAP. The participant received several prior lines of treatment including FOLFIRI+Bev (FIG. 13). In October 2020, the participant progressed on investigational drug and had increase in size of lung metastasis. In November 2020, the participant enrolled in EAP and received onvansertib 15 mg/m²+FOLFIRI+Bevacizumab. Clinical benefit and response to onvansertib+FOLFIRI+Bevacizumab combination were demonstrated. As shown in FIG. 14A, 8-week scans show decrease in size of numerous lung metastases (many appearing necrotic), and 16-week scan shows further decrease in size of lung metastases (many continuing to appear necrotic). Also, it was found that decreases in tumor lesions were accompanied by a decrease in KRAS MAF from 1.4% to 0% (undetectable) and a decrease in CEA from 24.4 to 4.6 ng/mL (FIG. 14B).

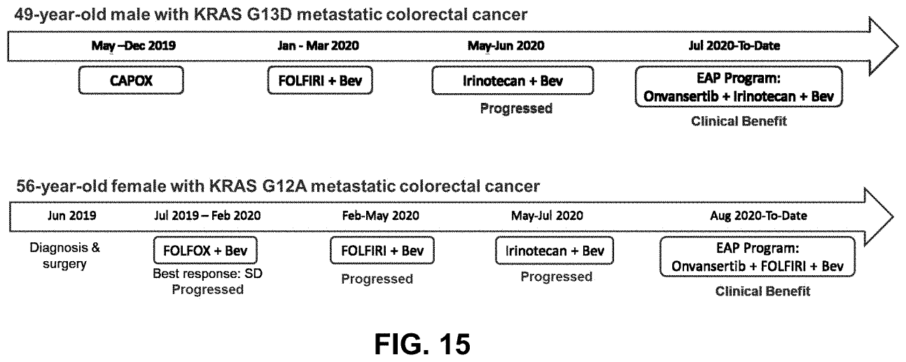

A 49-year-old male with KRAS G13D metastatic colorectal cancer and a 56-year-old female with KRAS G12A metastatic colorectal cancer participated in the EAP. Prior to EAP: both participants were progressing on irinotecan-based regimens (FIG. 15). Clinical benefit under EAP: both participants are showing clinical benefit with ongoing durable stable disease of 8 months (the 49-year-old male with KRAS G13D mCRC) and 7 months (56-year-old female with KRAS G12A mCRC), respectively.

Treatment with onvansertib+FOLFIRI+Bevacizumab in the EAP has been well tolerated with no SAEs reported to-date. At the cutoff date of Mar. 10, 2021, 20 participants with 3 or more prior therapies, were evaluated for clinical benefit. Median progression-free survival (PFS) was 5.6 months, and 11 participants remain on treatment (significant contrast with historical control of 2-3 months. Clinical benefit was observed in heavily pre-treated participants and those progressing on irinotecan-based regimens prior to enrolling in the EAP. Changes in plasma KRAS-mutant were found to correlate with clinical benefit. Participants with a greater than 50% decrease in KRAS mutant allelic frequency (MAF) had a significant increase in PFS compared with those who had a decrease of less than 50%.

### Example 3

### KRAS Mutations in Metastatic Castration-Resistant Prostate Cancer (mCRPC) Patients

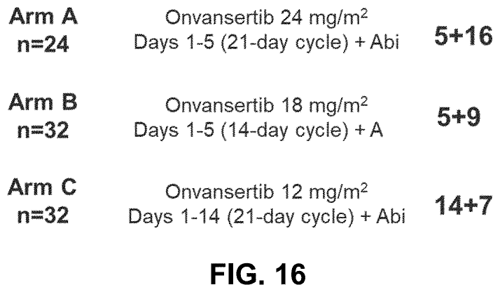

A Phase 2 Study of onvansertib, in combination with abiraterone and prednisone in Patients with mCRPC was conducted. The treatment schedule for Arms A, B and C is shown in FIG. 16. The enrollment as of Jan. 11, 2021 is shown in Table 5.

TABLE 5
Patient enrollment

| Number of patients (N) | Arm A | Arm B | Arm C |
|---|---|---|---|
| Treated | 24 | 17 | 10 |
| Completing 12-weeks | 14 | 8 | 6 |
| Currently on Treatment | 0 | 4 | 7 |

Key Eligibility Criteria:

Initial signs of abiraterone resistance defined as 2 rising PSAs; one rise of ≥0.3 ng/mL separated by one week

Key Exclusion Criteria:

(1) prior treatment with either enzalutamide or apalutamide, and (2) rapidly progressing disease or significant symptoms related to disease progression

Efficacy Endpoints:

Primary: Disease control evaluated as PSA decline or stabilization (PSA rise <25% over baseline) after 12 weeks of treatment. Secondary: Radiographic response per RECIST v1.1 criteria, time to PSA progression, and time to radiographic response.

Correlative Studies:

Analysis of circulating tumor cells (CTC), archival tissue, and circulating tumor DNA (ctDNA) to identify response biomarkers.

Baseline characteristics: shown in Table 6.

TABLE 6
Baseline characteristics

| Total patients N = 51 | Median [range] or n (%) | |
|---|---|---|
| Age, years | 72 | [51-87] |
| Nonwhite ethnicity | 7 | (14%) |
| ECOG | | |
| 0 | 43 | (84%) |
| 1 | 7 | (14%) |
| Years since diagnosis | 4 | [1-28] |
| Grade groups 4 and 5 | 29 | (57%) |
| De novo metastatic disease | 19 | (37%) |
| Presence of bone metastasis | 42 | (82%) |
| Presence of visceral metastasis | 18 | (35%) |
| Baseline PSA, ng/mL | 11.4 | [0.6-515] |
| AR-V7+ at baseline* | 10 | (20%) |
| Baseline CTC/7.5 mL of blood** | 15.8 | [0-653] |

*AR-V7 status was evaluated using the EPIC and Johns Hopkins University testing platforms
**CTC count was performed by EPIC

Safety Assessment:

Most frequent Grade 3 and 4 adverse events (AEs) were expected, on-target, reversible hematological (anemia, neutropenia, thrombocytopenia and WBC decrease), associated with the mechanism of action of onvansertib. Hematological AEs were reversible and effectively managed by dose delay, dose reduction and/or growth factor support. Table 7 shows most common treatment-emergent adverse events in treated patients (≥10% of patients)

TABLE 7
Most common treatment-emergent adverse events in treated patients

| | | | | | | All | |
|---|---|---|---|---|---|---|---|
| Adverse events | Grade 1 | | Grade 2 | Grade 3 | Grade 4 | grades | |
| Anemia | 10 | (20%) | 6 (12%) | 1 (2%) | | 17 | (33%) |
| Fatigue | 10 | (20%) | 3 (6%) | | | 13 | (25%) |
| Thrombocytopenia | 11 | (22%) | 1 (2%) | | | 13 | (25%) |
| Neutropenia | 1 | (2%) | 1 (2%) | 7 (14%) | | 12 | (24%) |
| Hypophosphatemia | 3 | (6%) | 3 (6%) | 4 (8%) | | 10 | (20%) |
| WBC decrease | 3 | (6%) | 2 (4%) | 3 (6%) | 2 (4%) | 10 | (20%) |
| Back pain | 4 | (8%) | 3 (6%) | | | 7 | (14%) |
| Hypokalemia | 3 | (6%) | 1 (2%) | 1 (2%) | | 5 | (10%) |

Efficacy:

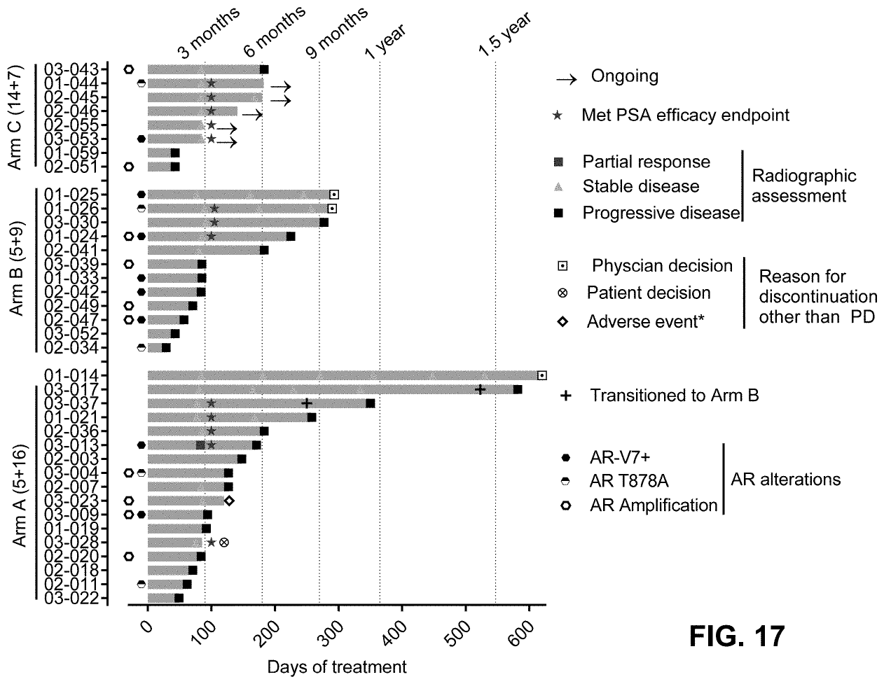

results are shown in Table 8 and FIG. 17. Nineteen (53%) patients had at least 1 AR alteration associated with abiraterone-resistance (AR-V7 expression, AR mutation T878A and/or amplification of AR). 5 (26%) patients had disease control at 12 weeks, and 8 (42%) patients had radiographic stable disease at 12 weeks.

TABLE 8
Efficacy results

| | Arm A | Arm B | Arm C |
|---|---|---|---|
| | (5 + 16) | (5 + 9) | (14 + 7) |
| Evaluable for efficacy* | 17 | 12 | 8 |
| Completed at least 12 weeks of treatment | 14 | 8 | 6 |
| Had radiographic or clinical progression | 3 | 4 | 2 |
| within 12 weeks | | | |
| Disease control at 12 weeks** | 5 (29%) | 3 (25%) | 5 (63%) |
| Radiographic stable disease at 12 weeks | 9 (53%) | 5 (42%) | 6 (75%) |
| Durable response (≥6 months) | 5 (29%) | 5 (42%) | 3 (37%) |

10 patients with unfavorable CTC count (≥5 CTC/7.5 mL of blood) at baseline were re-analyzed after 12 weeks of treatment: (1) 5 (50%) patients had a ≥80% CTC decrease including 4 who converted to favorable CTC level and 3 with no detectable CTC, (2) Median time on treatment was 9.2 months for patients with decrease CTC (n=5) vs 4.9 months for patients with increase CTC (n=5). Arms A (n=17) and B (n=12) showed similar efficacy with 29% and 25% of patients achieving the primary endpoint and 53% and 42% of patients with SD at 12 weeks, respectively. The more continuous dosing schedule of Arm C (n=8) has shown so far higher response rate with 63% of patients achieving the primary endpoint and 75% with SD at 12 weeks. Efficacy was observed in patients harboring AR alterations across all 3 arms. Onvansertib+abiraterone combination induced unfavorable-to-favorable CTC conversion, and this conversion was correlated with durable response.

Biomarker Analyses:

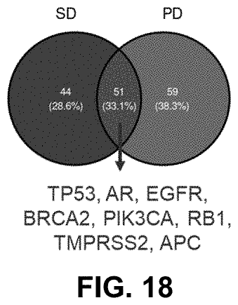

Circulating tumor dna (ctdna) genomic profiling was performed, in which mutation profiling of ctDNA isolated from baseline liquid biopsy was performed using Guardant platform. 33 patients were analyzed, and total of 379 somatic variants were identified in 154 genes, with a median number of variants of 9 [1-54] per patient. Genes differentially mutated in SD and PD patients were analyzed, including 18 patients had SD at 12 weeks (44 genes were exclusively mutated in SD patients, FIG. 18) and 15 patients had PD at or before 12 weeks (59 genes were exclusively mutated in PD patients, FIG. 18).

A gene list enrichment analysis tool (Enrichr) was used to compare lists of genes exclusively mutated in either SD or PD patients with the hallmark gene sets from the Molecular Signatures Database (MSigDB). Analysis showed enrichment for G2/M checkpoint, E2F target and DNA repair in SD, but not PD patients, consistent with the role of PLK1 in cell cycle regulation and DNA damage response pathways. Pathways enriched in SD patients are shown in Table 9, and pathways enriched in PD patients are shown in Table 10.

TABLE 9
Pathways enriched in SD patients (P-values < 0.05)
Pathways enriched in SD patients

Wnt-beta Catenin Signaling
PI3K/AKT/mTOR Signaling
G2-M Checkpoint
E2F Targets
IL-2/STAT5 Signaling
KRAS Signaling Up
IL-6/JAK/STAT3 Signaling
UV Response
DNA Repair
Apoptosis

TABLE 10
Pathways enriched in SD patients (P-values < 0.03)
Pathways enriched in PD patients

UV Response
Wnt-beta Catenin Signaling
KRAS Signaling Up
Apoptosis
Myogenesis
PI3K/AKT/mTOR Signaling
Notch Signaling
TNF-alpha Signaling via NF-kB
Apical Junction
IL-6/JAK/STAT3 Signaling

ctDNA analysis revealed differences in baseline genomic profiles of patients achieving SD at 12 weeks vs patients progressing before or at 12 weeks. Mutations exclusively present in SD patients were associated with cell cycle and DNA repair pathways that may result in increased sensitivity to onvansertib and efficacy of the combination. The results described herein indicate that a subset of patients with early resistance to abiraterone is more dependent on PLK1-related pathways and consequently more vulnerable to PLK1 inhibition.

### Example 4

### Monitoring KRAS Mutations for Determining Chemotherapy

KRAS monitoring is expected as an effective means of determining if a chemotherapy is to be maintained, removed, added back or modified in a cancer treatment using a PLK inhibitor (e.g., onvansertib). For example, based on monitoring of KRAS mutations in a cancer patient under treatment using onvansertib, chemotherapy can be removed from the treatment that the patient is receiving or be added back to the treatment.

A patient is receiving onvansertib, irinotecan, 5-FU and bevacizumab for cancer treatment. The patient has a positive clinical response which includes tumor shrinkage in scans and decreases in KRAS (e.g., >=75% decrease in KRAS mutation(s) from baseline at C2D1). The patient is monitored and if the KRAS remains >=75% at 6 months and the patient remains stable, irinotecan is removed and the patient goes on maintenance therapy which is onvansertib and oral 5-FU (with or without bevacizumab) and KRAS is continued to be monitored. If the KRAS mutations in the patient come back up to <75% of baseline after irinotecan is removed, irinotecan will be re-administered so that the patient is back on irinotecan+5-FU. This monitoring method has an advantage to allow the patient to “have a break” from irinotecan which causes various side effects, including fatigue.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of determining responsiveness of a subject to a cancer treatment, comprising
treating a subject with cancer, wherein the treating comprises administering a PLK1 inhibitor to the subject;
detecting change(s) in KRAS gene mutation(s) in the subject, and
determining the responsiveness of the subject to the cancer treatment based on the change(s) detected in KRAS gene mutation(s).

2. The method of claim 1, wherein detecting change(s) in KRAS gene mutation(s) in the subject comprises detecting one or more mutations in KRAS gene in the subject (1) during the subject is treated for cancer, (2) before the subject is treated for cancer, (3) after the subject is treated for cancer, or a combination thereof.

3. The method of any one of claims 1-2, wherein detecting change(s) in KRAS gene mutation(s) in the subject comprises detecting KRAS gene mutations two or more times in the subject, and optionally at least two of the two or more times occur within 5, 7, 14, 28, or 35 days.

4. The method of any one of claims 1-3, wherein change(s) in KRAS gene mutation(s) comprises (1) change(s) in KRAS gene mutation(s) during the subject is treated for cancer, (2) change(s) in KRAS gene mutation(s) from before the subject is treated for cancer to during the subject is treated for cancer, or a combination thereof.

5. The method of any one of claims 1-4, wherein detecting change(s) in KRAS gene mutation(s) comprises detecting variant allele frequency of KRAS gene.

6. The method of claim 5, wherein the variant allele frequency is mutant allelic frequency (MAF).

7. The method of any one of claims 4-6, wherein the variant allele frequency of KRAS gene is determined by total mutation count, mean variant allele frequency, number of KRAS mutation alleles per ml of plasma, or a combination thereof.

8. The method of any one of claims 1-7, detecting change(s) in KRAS gene mutation(s) in the subject comprises detecting change(s) in KRAS gene mutation(s) in a biological sample from the subject, or derivative thereof.

9. The method of claim 8, wherein the biological sample comprises a bodily fluid, whole blood, plasma, one or more tissues, one or more cells, or a combination thereof.

10. The method of claim 9, the bodily fluid comprises blood, plasma, urine, or a combination thereof.

11. The method of any one of claims 8-10, wherein the biological sample comprises circulating tumor DNA (ctDNA), cell-free DNA (cfDNA), circulating tumor cell (CTC), or a combination thereof.

12. The method of claim 11, comprising analyzing the ctDNA using polymerase chain reaction (PCR) or next generation sequencing (NGS), and wherein the PCR is optionally droplet digital PCR (ddPCR).

13. The method of any one of claims 1-12, wherein the subject has one or more mutations in KRAS gene before being treated with the PLK1 inhibitor.

14. The method of any one of claims 1-12, wherein the subject does not have mutations in KRAS gene before being treated with the PLK1 inhibitor.

15. The method of any one of claims 1-14, wherein determining the responsiveness of the subject comprises determining if the subject is a responder of the treatment, if the subject is or is going to be in complete recover (CR), or if the subject is or is going to be in partial remission (PR).

16. The method of any one of claims 1-14, wherein determining the responsiveness of the subject comprises determining progression-free survival (PFS) of the subject.

17. The method of any one of claims 1-14, wherein determining the responsiveness of the subject comprises determining if the subject has a partial response to the treatment, if the subject has a complete response to the treatment, if the subject has a stable disease (SD) status, or if the subject has a progressive disease (PD) status.

18. The method of any one of claims 1-17, wherein the KRAS mutation is measured by determining the amount of the KRAS mutations in the sample, determining the amount of the KRAS mutation in proportion to the amount of total KRAS in the sample, or both.

19. The method of any one of claims 6-18, wherein the cancer treatment with the PLK1 inhibitor is maintained if the change in MAF of KRAS is a decrease of at least 25%, at least 50%, or at least 75%, and optionally the decrease is detected at the end of cycle 1 of the cancer treatment or at day 1 of cycle 2 of the cancer treatment.

20. The method of any one of claims 6-19, wherein the cancer treatment is for at least one month, at least three months, or at least six months.

21. The method of any one of claims 6-20, wherein the cancer treatment comprises a chemotherapy and the cancer treatment is modified to remove the chemotherapy partially or completely if the change in MAF of KRAS is a decrease of at least 50% or at least 75% after receiving the cancer treatment for six months.

22. The method of claim 21, further comprising measuring KRAS mutation after partial or complete removal of the chemotherapy, and restoring the chemotherapy if the KRAS mutation level increases compared to the KRAS mutation level at the time of the removal of the chemotherapy.

23. The method of any one of claims 19-20, the decrease is detected at the end of cycle 1 of the cancer treatment or at day 1 of cycle 2 of the cancer treatment.

24. The method of any one of claims 1-18, wherein the cancer treatment with the PLK1 inhibitor is maintained if KRAS mutation in the samples decreases to below 0.01% or below 0.001% of KRAS in the sample.

25. The method of any one of claims 6-18, wherein the cancer treatment with the PLK1 inhibitor is modified or discontinued if the change in MAF of KRAS is a decrease of less than 50%, less than 25%, or less than 10%, and optionally the decrease is detected at the end of cycle 1 of the cancer treatment or at day 1 of cycle 2 of the cancer treatment.

26. The method of any one of claims 6-18, wherein the cancer treatment does not comprise a chemotherapy and the cancer treatment is modified to add a chemotherapy if the change in MAF of KRAS is a decrease of less than 50% or less than 75%.

27. The method of any one of claims 21, 22 and 26, the chemotherapy comprises irinotecan, and optionally the chemotherapy is FOLFIRI.

28. The method of any one of claims 1-18, wherein the cancer treatment with the PLK1 inhibitor is modified or discontinued if KRAS mutation in the samples does not decrease to below 0.01% or below 0.001% of KRAS in the sample.

29. The method of any one of claims 1-28, wherein detecting change(s) in KRAS gene mutation(s) in the subject comprising detecting one or more KRAS mutations emerged in the subject after the subject being treated with the PLK1 inhibitor.

30. A method of improving outcome of a cancer treatment, comprising
detecting variant allele frequency of KRAS gene in a subject at a first time point in a first sample, wherein the first time point is before the subject starts the cancer treatment, or during the cancer treatment, and wherein the cancer treatment comprises administering a PLK1 inhibitor to the subject;
detecting variant allele frequency in KRAS gene in the subject at one or more additional time points in one or more additional samples after the subject, wherein the at least one of the one or more additional time points is during the cancer treatment;
determining the difference of the variant allele frequency of KRAS between the first and the one or more additional samples, wherein a decrease in the variant allele frequency in at least one of the one or more additional samples relative to the first sample indicates the subject as responsive to the cancer treatment; and
continuing the cancer treatment to the subject if the subject is indicated as responsive to the cancer treatment, or discontinuing the cancer treatment to the subject and/or starting a different cancer treatment to the subject if the subject is not indicated as responsive to the cancer treatment.

31. The method of claim 30, wherein the first time point is before the subject starts the cancer treatment.

32. The method of any one of claims 30-31, wherein at least two of the additional time points are during the cancer treatment.

33. A method of treating cancer, comprising:
treating a subject with cancer, wherein the treating comprises administering a PLK1 inhibitor to the subject;
determining a decrease, relative to a variant allele frequency of KRAS gene or number of KRAS mutant copy per unit in a first sample of the subject obtained at a first time point before the subject receives the cancer treatment or during the cancer treatment, in a variant allele frequency of KRAS gene in a second sample of the subject obtained at a second time point after the subject starts receiving the cancer treatment; and
continuing with the cancer treatment.

34. The method of any one of claims 30-33, wherein the first time point is prior or immediately prior to the cancer treatment.

35. The method of any one of claims 30-33, wherein the first time point is during the cancer treatment, and optionally at day 5, 7, 14, or 28 of the cancer treatment.

36. The method of any one of claims 30-35, wherein the one or more additional time points are during the cancer treatment, and optionally at day 5, 7, 14, 28, or 35 of the cancer treatment.

37. The method of any one of claims 30-36, wherein the first time point and at least one of the one or more additional time points are during the first cycle of the cancer treatment.

38. The method of any one of claims 30-37, wherein at least one of the one or more additional time points are during the first cycle of the cancer treatment, and at least one of the one or more additional time points are during the second cycle of the cancer treatment.

39. The method of any one of claims 30-38, wherein the variant allele frequency is mutant allelic frequency (MAF).

40. The method of, wherein the determining step comprises determining a decrease in the number of mutant copies per unit of the first sample and/or the second sample, wherein the unit is optionally ml, and optionally the first sample and/or the second sample is a plasma sample.

41. The method of any one of claims 30-40, wherein the variant allele frequency of KRAS gene is determined by total mutation count, mean variant allele frequency, number of KRAS mutation alleles, or a combination thereof.

42. The method of any one of claims 1-40, detecting variant allele frequency in KRAS gene comprises detecting variant allele frequency in KRAS gene in a biological sample from the subject, or derivative thereof.

43. The method of claim 42, wherein the biological sample comprises a bodily fluid, whole blood, plasma, one or more tissues, one or more cells, or a combination thereof.

44. The method of claim 43, the bodily fluid comprises blood, plasma, urine, or a combination thereof.

45. The method of any one of claims 42-44, wherein the biological sample comprises circulating tumor DNA (ctDNA), circulating tumor cell (CTC), or a combination thereof.

46. The method of claim 45, comprising analyzing the ctDNA using polymerase chain reaction (PCR) or next generation sequencing (NGS), and wherein the PCR is optionally droplet digital PCR (ddPCR).

47. The method of any one of claims 1-46, wherein the subject has one or more mutations in KRAS gene before being treated with the PLK1 inhibitor.

48. The method of any one of claims 1-46, wherein the subject does not have mutations in KRAS gene before being treated with the PLK1 inhibitor.

49. The method of any one of claims 1-48, wherein the subject has received one or more prior cancer treatment.

50. The method of any one of claims 1-49, wherein the cancer is advanced, metastatic, refractory, or relapsed.

51. The method of any one of claims 1-50, wherein the cancer is colorectal cancer, pancreatic cancer, leukemia, lung cancer, or a combination thereof.

52. The method of any one of claims 1-51, wherein the cancer is a KRAS mutation cancer.

53. The method of any one of claims 1-52, wherein the cancer is colorectal cancer, optionally metastatic colorectal cancer.

54. The method of any one of claims 1-53, wherein the KRAS gene mutation(s) comprise mutations at codon 12, codon 13, codon 18, codon 61, codon 117, codon 146, or a combination thereof.

55. The method of any one of claims 1-53, wherein the KRAS gene mutation(s) comprise mutations at codon 12 and/or codon 13.

56. The method of any one of claims 1-53, wherein the KRAS gene mutation(s) comprise G12A, G12C, G12D, G12R, G12S, G12V, G13C, G13D, G13S, G13R, A18D, G61H, Q61L, Q61K, Q61R, K117N, A146T, A146V, A146P, A11V, or a combination thereof.

57. The method of any one of claims 1-56, wherein the PLK1 inhibitor is onvansertib, BI2536, volasertib (BI 6727), GSK461364, HMN-176, HMN-214, AZD1775, CYC140, rigosertib (ON-01910), MLN0905, TKM-080301, TAK-960, Ro3280, or a combination thereof.

58. The method of any one of claims 1-56, wherein the PLK1 inhibitor is onvansertib.

59. The method of claim 58, wherein the treatment comprises administration of onvansertib every day in a cycle of 28 days.

60. The method of claim 58, wherein the treatment comprises administration of onvansertib for the first 21 days and not the last 7 days in a cycle of 28 days.

61. The method of claim 58, wherein the treatment comprises administration of onvansertib for ten days in a cycle of 28 days.

62. The method of claim 58, wherein the treatment comprises administration of onvansertib for five days in the first 14 days and five days in the second 14 days in a cycle of 28 days.

63. The method of any one of claims 58-62, wherein the treatment comprises administration of onvansertib at 6 mg/m²-24 mg/m², optionally 6 mg/m²-12 mg/m² or 12 mg/m²-18 mg/m².

64. The method of any one of claims 58-63, wherein a maximum concentration (Cₘₐₓ) of onvansertib in a blood of the subject is from about 100 nmol/L to about 1500 nmol/L.

65. The method of any one of claims 58-64, wherein an area under curve (AUC) of a plot of a concentration of onvansertib in a blood of the subject over time is from about 1000 nmol/L·hour to about 400000 nmol/L·hour.

66. The method of any one of claims 58-65, wherein a time (Tmax) to reach a maximum concentration of onvansertib in a blood of the subject is from about 1 hour to about 5 hours.

67. The method of any one of claims 58-66, wherein an elimination half-life (T_{1/2}) of onvansertib in a blood of the subject is from about 10 hours to about 60 hours.

68. The method of any one of claims 1-67, wherein the cancer treatment comprises administering to the subject at least one additional cancer therapeutics or cancer therapy.

69. The method of claim 68, wherein the additional cancer therapeutics comprises FOLFIRI, bevacizumab, abiraterone, FOLFOX, an anti-EGFR agent, a KRAS directed inhibitor, gemcitabine, abraxane, nanoliposomal irinotecan, 5-FU, or a combination thereof; wherein the anti-EGFR agents is optionally cetuximab, and KRAS directed inhibitor is optionally a G12C inhibitor, a G12D inhibitor or a combination thereof.

70. The method of any one of claims 68-69, wherein the PLK inhibitor and the cancer therapeutics or cancer therapy are co-administered simultaneously or sequentially.

71. The method of any one of claims 1-70, wherein the cancer treatment comprises one or more cycles, and change(s) in KRAS gene mutation(s) or variant allele frequency of KRAS is detected before, during and/or after each cycle of the cancer treatment.

72. The method of claim 71, wherein each cycle of treatment is at least 21 days.

73. The method of claim 71, wherein each cycle of treatment is from about 21 days to about 28 days.

74. The method of any one of claims 1-73, wherein the subject is human.

75. Use of a PLK1 inhibitor as a treatment of a subject with cancer, wherein the responsiveness of the subject to the treatment is determined using a method of any one of claims 1-29.

76. Use of a PLK1 inhibitor as a treatment of a subject with cancer, wherein the treatment outcome is improved using a method of any one of claims 30-32 and 34-74.

77. Use of a PLK1 inhibitor as a treatment of a subject with cancer, wherein the subject is treated using a method of any one of claims 33-74.

78. The use of any one of claims 75-77, wherein the PLK1 inhibitor is onvansertib.
